(19) United States
(12) Patent Application Publication
(10) Pub. No.: US 2024/0042093 A1
(43) Pub. Date: Feb. 8, 2024

(54) IN SITU SOLIDIFYING INJECTABLE COMPOSITIONS WITH TRANSIENT CONTRAST AGENTS AND METHODS OF MAKING AND USING THEREOF

(71) Applicant: FLUIDX MEDICAL TECHNOLOGY, INC., Salt Lake City, UT (US)

(72) Inventors: Russell J. Stewart, SALT LAKE CITY, UT (US); Joshua JONES, SANDY, UT (US); Shawn FOJTIK, Park City, UT (US); Jessica KARZ, Salt Lake City, UT (US)

(21) Appl. No.: 18/268,786

(22) PCT Filed: Dec. 22, 2021
(86) PCT No.: PCT/US21/64806

§ 371 Date: Jun. 21, 2023

Related U.S. Application Data
(60) Provisional application No. 63/129,162, filed on Dec. 22, 2020.

(51) Int. Cl. A61L 24/00; A61L 24/06

(52) U.S. Cl. CPC A61L 24/0015; A61L 24/06; A61L 2300/106; A61L 2300/44; A61L 2400/06

(57) ABSTRACT

Described herein are injectable compositions composed of one or more polycationic polyelectrolytes and anionic counterions, one or more one polyanionic polyelectrolytes and cationic counterions, and a transient contrast agent. The injectable compositions have an ion concentration that is sufficient to prevent association of the polycationic polyelectrolytes and the polyanionic poly-electrolytes in water. Upon introduction of the composition into a subject, a solid is produced in situ. The transient contrast agent diffuses out of the solid over hours or days providing temporary contrast and does not remain in the subject unlike permanent contrast agents. This feature provides sufficient time for the clinician to perform medical procedures prior to the diffusion of the contrast agent out of the solid. The viscosity of the injectable compositions can be varied depending upon the application of the injectable composition. By varying the molecular weight, charge densities, and/or concentrations of the polycationic and polyanionic salts, it is possible to produce injectable compositions having a useful range of viscosities.

### CROSS-REFERENCE TO RELATED APPLICATIONS

This application claims the benefit of and priority to co-pending U.S. Provisional Patent Application No. 63/129,162, filed on Dec. 22, 2020, the contents of which are incorporated by reference herein in their entireties.

### BACKGROUND

Transcatheter embolization is a medical procedure used to occlude a blood vessel or vascular bed. In this procedure, vascular access is obtained, typically in the femoral artery, and the catheter is guided into position using fluoroscopy. An embolization agent is delivered to produce a controlled, localized blockage. Embolization therapy is widely employed in the treatment algorithms for an array of conditions. Embolic devices are used as a primary mode of therapy to treat certain types of hemorrhage, including upper and lower gastrointestinal bleeding [1-3], pulmonary and bronchial hemorrhage [4, 5], subdural hematomas [6, 7], and pelvic hemorrhage [8]. Vascular abnormalities such as arteriovenous malformations [9, 10], fistulas [11], aneurysms [12], and varicoceles are also commonly treated using embolization. Benign tumors (e.g. uterine fibroids) and malignant tumors, such as hepatocellular carcinoma[14, 15], head and neck cancer [16], and renal cell carcinoma are targets for embolization. In the latter case, this is most often a palliative treatment, but embolization can be done prior to resection to minimize bleeding during surgery [14, 18]. Furthermore, pre-operative embolization of the portal vein is commonly done to stimulate hypertrophy in lobes of the liver not destined for removal [19, 20]. In addition to these well-established uses, embolization is being explored within the treatment algorithms in several new indications including the treatment of benign prostatic

hyperplasia (prostatic artery embolization) [21], obesity (bariatric embolization) [22], and osteoarthritis [23].

To perform an embolization procedure, a variety of embolic devices or agents can be deployed, depending on the size of vessel to be occluded [24, 25]. Large vessels (>1 mm) are typically occluded by using thrombogenic occlusion devices such as coils

or vascular plugs [24]. Smaller vessels are occluded with microspheres and embolic particles, ranging in size from 40-1200 μm, which are carried downstream from the catheter by blood flow and become lodged in vessels.

Liquid embolic agents have a low viscosity injectable form, allowing their delivery through long microcatheters, but harden upon entering blood vessels. These agents are most often used in situations where distal penetration into smaller vessels (<300 μm) is desired [25]. Classes of liquid embolic agents in clinical use include precipitating ethylene-vinyl copolymers (EVOH) and in situ polymerizing cyanoacrylate (CA) glues. EVOH-based embolics (e.g. Onyx™, PHIL™, Squidperi™) contain polymers dissolved in dimethyl sulfoxide (DMSO) that precipitate in situ as the DMSO diffuses away and cyanoacrylate glues (e.g. Trufill™) that polymerize upon contact with anions in blood [27].

Fluoroscopic visualization during delivery is an essential characteristic of an embolization procedure. The type of visualization agent used with a liquid embolic is an important design criterion. Permanent radiopacity provides advantages such as high contrast imaging of the injection site both during and after the procedure. However, these agents remain in the subject indefinitely and can cause imaging artifacts in CT scans, as well as provide undesirable discoloration under the skin where the contrast agent is located. Furthermore, permanent contrast agents such as tantalum can undergo sparking if electrocautery is subsequently performed at the injection site. Conversely, contrast that is immediately washed away from the embolization site, such as when beads or particles are delivered in a contrast medium carrier, does not allow the clinician to visualize the position of the first beads or particles if additional injections are necessary. It is therefore desirable to have temporary contrast that persists for at least the length of the medical procedure, but that disappears within hours or days to avoid the disadvantages of permanent contrast agents.

Another critical design criteria of liquid embolics is the viscosity of the composition. Embolic agents are generally administered through long, narrow microcatheters. Small internal diameter (i.d.) microcatheters require low viscosity compositions to achieve practical injection pressures, e.g., below the microcatheter burst pressure. Higher viscosities are appropriate for larger i.d. microcatheters for embolizing larger blood vessels. Liquid embolics with viscosities optimized for a range of microcatheter dimensions, which still achieve effective and precise embolization, is of great value to clinicians.

A clinical need exists for liquid embolic agents that include contrast agents that provide temporary contrast for minutes to hours rather than immediately diffusing from the embolic once administered to the subject or remain in the subject permanently, and that are available in a range of viscosities suited to the mode and site of administration of the embolic to the subject.

### SUMMARY

Described herein are injectable compositions composed of one or more polycationic polyelectrolytes and anionic counterions, one or more one polyanionic polyelectrolytes and cationic counterions, and a transient contrast agent. The injectable compositions have an ion concentration that is sufficient to prevent association of the polycationic polyelectrolytes and the polyanionic polyelectrolytes in water. For example, the counterions are of sufficient concentration to prevent the polycations and polyanions from associating electrostatically, which results in the formation of a stable injectable composition. Upon introduction of the composition into a subject, a solid is produced in situ. The transient contrast agent diffuses out of the solid over hours or days providing temporary contrast and does not remain in the subject unlike permanent contrast agents. This feature provides sufficient time for the clinician to perform medical procedures prior to the diffusion of the contrast agent out of the solid. The viscosity of the injectable compositions can be varied depending upon the application of the injectable composition. By varying the molecular weight, charge densities, and/or concentrations of the polycationic and polyanionic salts, it is possible to produce injectable compositions having a useful range of viscosities.

The advantages of the invention will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows the maximum deliverable viscosity as a function of catheter internal diameter, assuming a catheter burst pressure of 800 psi and length of 150 cm, as predicted by Poiseulle's law.

FIG. 2 shows the structures of exemplary iodinated contrast agents.

FIGS. 3A-3B show the effect of polymer concentration and molecular weight (M_{w}) on viscosity of injectable compositions comprising the polyelectrolytes poly(GPMA·HClₙ-co-MA) (PG-HClₙ) and sodium hexametaphosphate (NaₙMP) at a fixed polyelectrolyte positive to negative charge ratio of 1:1: Viscosity (y-axis) is plotted vs. PG-HClₙ concentrations (x-axis) at different PG concentrations.

FIG. 4 shows the viscosity of injectable compositions prepared with PG-HClₙ and NaₙMP vs. concentration (mg I/mL) of non-ionic contrast media (Iohexol and Iodixanol).

FIG. 5 shows an injectable composition with iodixanol (80 mgI/mL) being delivered into saline and transitioning into the solid form.

FIG. 6 shows the effect of molecular weight, polymer concentration, and added counterions on the modulus of the solidified injectable compositions 24 hours after injection into normal saline. Oscilatory storage modulus values are shown at 1 Hz., 1% strain.

FIG. 7 shows comparison of the complex modulus (G*) of the liquid and solid forms of PG-MP injectable compositions prepared with non-ionic contrast on a log scale. G* values are reported at 1 Hz, 1% strain.

FIGS. 8A-8B show the duration of radiopacity for injectable compositions with varying concentrations of Iodixanol. Panel A shows radiopacity measured in Hounsfield units at 1 hour post-delivery and 24 hours post-delivery for injectable compositions with iohexol concentrations ranging from 0 mgI/mL to 320 mgI/mL. Panel B shows images from two of these samples (80 mg/mL and no contrast) in vertical and axial images at 1 and 24 hours. Radiopacity is markedly decreased in all samples at 24 hours.

FIGS. 9A-9B show the use of the injectable composition (IC) prepared with iohexol (300 mgI/mL) in a swine kidney. (A) Image taken within 5 minutes of delivery showing radiopacity in the area of the IC-Iohexol 300 delivery. (B) Image taken approximately 24 hours after delivery showing no remaining radiopacity in the area where IC-Iohexol 300 was delivered, demonstrating the transient nature of the contrast.

FIGS. 10A-10D show the use of the injectable composition with ethiodized oil (1:1) in a swine kidney. (A) A pretreatment angiogram showing the arterial vasculature of the swine kidney. (B) Fluoroscopic image showing delivery of the IC-Ethiodized Oil emulsion. (C) A post-treatment angiogram taken within 5 minutes of delivery showing complete occlusion of the targeted vasculature. (D) 24 hour fluoroscope image of left kidney, showing no remaining contrast for the injectable composition.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

In the specification and in the claims that follow, reference will be made to a number of terms that shall be defined to have the following meanings:

It must be noted that, as used in the specification and the appended claims, the singular forms “a,” “an,” and “the” include plural referents unless the context clearly dictates otherwise. Thus, for example, reference to a “polycationic salt” includes mixtures of two or more such polycationic salts, and the like.

“Optional” or “optionally” means that the subsequently described event or circumstance can or cannot occur, and that the description includes instances where the event or circumstance occurs and instances where it does not. For example, the phrase “optionally comprises a reinforcing agent” means that the reinforcing agent can or cannot be included in the compositions and that the description includes both compositions including the reinforcing agent and excluding the reinforcing agent.

Throughout this specification, unless the context dictates otherwise, the word “comprise,” or variations such as “comprises” or “comprising,” will be understood to imply the inclusion of a stated element, integer, step, or group of elements, integers, or steps, but not the exclusion of any other element, integer, step, or group of elements, integers, or steps.

As used herein, the term “about” is used to provide flexibility to a numerical range endpoint by providing that a given numerical value may be “a little above” or “a little below” the endpoint without affecting the desired result. For purposes of the present disclosure, “about” refers to a range extending from 10% below the numerical value to 10% above the numerical value. For example, if the numerical value is 10, “about 10” means between 9 and 11 inclusive of the endpoints 9 and 11.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight of component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included. Weight percent includes and covers weight/volume percent and weight/weight percent.

The term “alkyl group” as used herein is a branched or unbranched saturated hydrocarbon group of 1 to 25 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl, and the like. Examples of longer chain alkyl groups include, but are not limited to, a palmitate group. A “lower alkyl” group is an alkyl group containing from one to six carbon atoms.

The term “cycloalkyl group” as used herein is a non-aromatic carbon-based ring composed of at least three carbon atoms. Examples of cycloalkyl groups include, but are not limited to, cyclopropyl, cyclobutyl, cyclopentyl, cyclohexyl, etc.

The term “treat” as used herein is defined as maintaining or reducing the symptoms of a pre-existing condition when compared to the same symptoms in the absence of the injectable composition. The term “prevent” as used herein is the ability of the injectable compositions described herein to completely eliminate the activity or reduce the activity when compared to the same activity in the absence of the injectable composition. The term “inhibit” as used herein refers to the ability of the injectable composition to slow down or prevent a process.

“Subject” refers to mammals including, but not limited to, humans, non-human primates, sheep, dogs, rodents (e.g., mouse, rat, guinea pig, etc.), cats, rabbits, cows, horses, and non-mammals including vertebrates, birds, fish, amphibians, and reptiles.

The term “salt” as used herein is defined as a dry solid form of a water-soluble compound that possesses cations and anions. When the salt is added to water, the salt dissociates into cations and anions. A polycationic salt is a compound having a plurality of cationic groups with anionic counterions. A polyanionic salt is a compound having a plurality of anionic groups with cationic counterions.

The term “polyelectrolytes” as used herein is defined as polymers with ionized functional groups, where the ionized functional groups can incorporated in the polymer backbone, a sidechain of the polymer, or a combination thereof. Polycations and polyanions are produced when a polycationic salt or a polyanionic salt is dissolved in water.

The term “molecular weight” is used herein to refer to the average molecular mass of an ensemble of synthetic polymers that contains a distribution of molecular masses. Unless otherwise noted, values reported herein are weight-average molecular weight (Mw).

The term “stable solution” as used herein is defined as a liquid composition of oppositely charged polyelectrolytes that do not interact electrostatically. The polyelectrolyte solutions do not separate into macroscopically distinct phases.

The term “solid” as used herein is defined as a non-fluid, viscoelastic material that has a substantially higher elastic modulus and viscous modulus than the initial fluid form of the injectable composition used to produce the solid.

The term “transient” as used herein with respect to the contrast agent is defined herein as the ability of the contrast agent to diffuse or escape over time the solid produced by the injectable compositions described herein.

The term “temporary contrast” as used herein occurs when the majority of the transient contrast agent diffuses from the solid such that the transient contrast agent cannot be detected in the subject by imaging techniques such as, for example, fluoroscopy or CT.

The term “critical ion concentration” is the concentration of ions above which a specific combination of polycations and polyanions do not associate electrostatically, thus preventing liquid-liquid or liquid-sold phase separation. The critical ion concentration for a specific composition depends on multiple factors, including the molecular weight and concentration of the polyelectrolyte pairs, the mol % of polymeric ions, the polymeric ion species, the free ion species, and pH. The counterions that dissociate from the polymeric salts upon dissolution in water contribute to the total ion concentration of the solution. In most cases, for the polyelectrolyte pairs and concentrations described herein, the concentration of dissociated counterions is above the critical ion concentration for the specific composition. In some cases, additional ions (e.g., monovalent ions such as NaCl) can be added to increase the total ion concentration to above the critical ion concentration for the specific composition.

“Physiological conditions” refers to conditions such as osmolality, ion concentrations, pH, temperature, etc. within a particular area of the subject. For example, the normal blood sodium concentration range is between 135 and 145 mMol/L in a human.

As used herein, a plurality (i.e., more than one) of items, structural elements, compositional elements, and/or materials may be presented in a common list for convenience. However, these lists should be construed as though each member of the list is individually identified as a separate and unique member. Thus, no individual member of any such list should be construed as a de facto equivalent of any other member of the same list based solely on its presentation in a common group, without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range was explicitly recited. As an example, a numerical range of “about 1” to “about 5” should be interpreted to include not only the explicitly recited values of about 1 to about 5, but also to include individual values and sub-ranges within the indicated range. Thus, included in this numerical range are individual values such as 2, 3, and 4, the sub-ranges such as from 1-3, from 2-4, from 3-5, from about 1-about 3, from 1 to about 3, from about 1 to 3, etc., as well as 1, 2, 3, 4, and 5, individually. The same principle applies to ranges reciting only one numerical value as a minimum or maximum. Furthermore, such an interpretation should apply regardless of the breadth or range of the characters being described.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc., of these materials are disclosed, that while specific reference of each various individual and collective combination and permutation of these compounds may not be explicitly disclosed, each is specifically contemplated and described herein. For example, if a class of molecules A, B, and C are disclosed, as well as a class of molecules D, E, and F, and an example of a combination A+D is disclosed, then even if each is not individually recited, each is individually and collectively contemplated. Thus, in this example, each of the combinations A+E, A+F, B+D, B+E, B+F, C+D, C+E, and C+F, are specifically contemplated and should be considered disclosed from disclosure of A, B, and C; D, E, and F; and the example combination of A+D. Likewise, any subset or combination of these is also specifically contemplated and disclosed. Thus, for example, the sub-group of A+E, B+F, and C+E is specifically contemplated and should be considered disclosed from disclosure of A, B, and C; D, E, and F; and the example combination of A+D. This concept applies to all aspects of this disclosure including, but not limited to, steps in methods of making and using the disclosed compositions. Thus, if there exist a variety of additional steps that can be performed with any specific embodiment or combination of embodiments of the disclosed methods, each such combination is specifically contemplated and should be considered disclosed.

### Injectable In Situ Solidifying Injectable Compositions with Transient Contrast Agents

Described herein are injectable compositions produced by mixing at least one polycationic salt, at least one polyanionic salt, and a contrast agent in water. Upon addition to water, the polycationic salt and polyanionic salt dissociate to produce a solution of polycations, polyanions, and counterions. The concentration of the counterions in solution is greater than the critical ion concentration of the composition, which is sufficient to prevent electrostatic association and subsequent separation of the polyelectrolytes into distinct liquid or solid phases. The application site within a subject has total ion concentrations below the ion concentration of the injectable composition, resulting in polyelectrolyte association and formation of a solid upon administration of the injectable composition into the subject.

Upon introduction of the injectable composition into the subject (e.g., within a blood vessel), the counterions present in the injectable composition diffuse out from the composition. Diffusion of ions out of the injectable composition allows electrostatic interactions between polycations and polyanions present in the composition, resulting in conversion of the polyelectrolytes into a non-fluid, water-insoluble solid in situ. The solid produced in situ is a stiff cohesive material that remains positioned at the site of solidification within the subject.

The injectable compositions described herein have numerous advantages over previous established embolics. The transient contrast agents present in the injectable compositions described herein readily diffuse from the solid produced in situ upon administration to the subject. The transient contrast agents permit facile imaging of the solid produced in situ at the time of administration of the injectable composition; however, the majority if not all of the transient contrast agent diffuses from the solid over a period of time. In contrast to other embolic agents with immediate or short-term radiopacity, where the agent diminishes in seconds after administration to a subject, the transient contrast agent in the solid produced by the injectable compositions described herein remain in the solid for a period of hours. In other words, there is contrast of an intermediate duration between rapidly dissipating contrast agents and permanent contrast agents; release of the transient contrast agent from the solid is delayed over an extended period of time. This feature permits the delivered embolic to remain visible throughout the duration of the embolization procedure, which results in better confirmation of material placement as well as provide guidance for subsequent injections during the procedure. This temporary radiopacity or contrast provides utility in that it does not interfere in any subsequent imaging, including fluoroscopy or CT, or future treatment of nearby targets. It also allows electrocautery to be used without sparking, in contrast to liquid embolization agents with metallic contrast. Thus, the injectable compositions described herein thus address the shortcomings regarding the use of permanent contrast agents.

Another advantage of the injectable compositions is the viscosity of the composition can be modified or fine-tuned depending upon the application of the injectable composition. As will be discussed in detail below, varying parameters such as, for example, the concentration and/or molecular weight of the polycationic salt and polyanionic salt can be used to modify the viscosity of the composition. Furthermore, the concentration of the transient contrast agent can also be used to modify the viscosity of the composition. This makes the injectable compositions versatile in a number of different applications, as the injectable compositions can be administered using needles, catheters, microcatheters or other delivery devices having a wide range of internal diameters and lengths that require the use of injectable compositions having different viscosities.

Another critical design criteria of liquid embolics is the viscosity of the composition. Viscosity determines the size of microcatheter through which an embolic can be delivered. A key factor in the ability to deliver a liquid embolic is the burst pressure of the microcatheter, the highest hydrodynamic pressure it can withstand as the fluid is pushed through the catheter. This pressure is determined and specified for each commercial microcatheter. These burst pressures vary from 300 psi to 1200 psi, but 800 psi is a common value for high-end embolic microcatheters. A variety of factors influence the maximum hydrodynamic pressure on the catheter. These factors are related by Poiseuille's equation where P is pressure, r is the radius of the tube (catheter), L is the length of the catheter, Q is the volumetric flow rate, and μ is viscosity of the embolic. This equation assumes a steady laminar flow through a cylindrical tube, which are generally appropriate for this application.

$$Poiseulle ’ s equation : Δ P = \frac{8 Q μ L}{π {r}^{4}}$$

This equation predicts maximum hydrodynamic pressure within the catheter, assuming pressure at the end of the catheter is at or reasonably close to zero and Newtonian fluid behavior. As a result, the burst pressure of the catheter constrains properties of the embolic agent, catheter, and delivery rate. The most consequential of these factors is the internal radius of the catheter since it is related to pressure by the inverse fourth power, meaning that decreasing the catheter radius by half increases the hydrodynamic pressure 16-fold. While careful selection of catheter size is important for successful embolization, it is in many ways limited by the specific application. For example, many situations require directing catheters into blood vessels less than 1 mm in diameter, necessitating the use of catheters <3 F (1 mm) in outer diameter. These catheters have internal diameters no greater than 0.027″ (0.69 mm). Some highly selective or neurovascular applications require catheters less than 2 F in outer diameter, which have internal diameters less than 0.014″ (0.36 mm). Given the limitations of controlling catheter diameter, control of other parameters is required to ensure successful application. Other factors within the equation that are directly proportional to hydrodynamic pressure are catheter length, flow rate of the material, and viscosity of the material. Length of the catheter and flow rate of material are properties are also largely governed by the procedure specifics or operator preference, leaving viscosity of the material as the primary factor for controlling injectability.

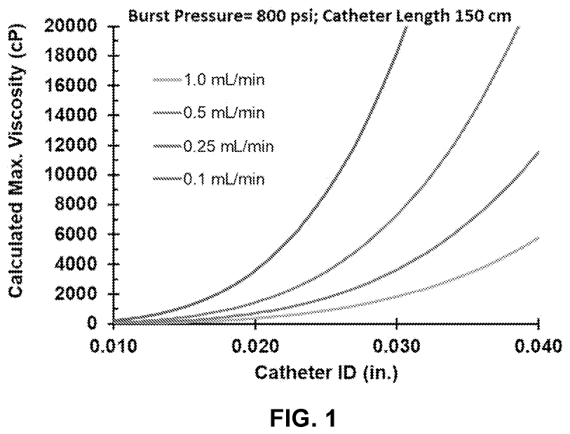

FIG. 1 illustrates the impact of fluid viscosity and catheter size on deliverability of a liquid. In this figure, maximum deliverable viscosity is plotted as a function of catheter internal diameter (ID) at flow rates ranging from 0.1-1 mL per minute. For this figure, catheter burst pressure is fixed at 800 psi (a common burst pressure for high-quality embolic microcatheters and length is fixed at 150 cm. In practice, a range of catheter lengths (˜100 cm-200 cm) and burst pressures (˜300 psi-1200 psi) can be found, but maximum viscosity scales linearly with both. For large catheters (≥0.040″ ID), catheters, viscosities greater than 5000 cP are acceptable even at the high flow rate of 1.0 mL/min, and viscosities higher than 10,000 cP can be delivered at 0.5 mL/min. However, as catheter size is decreased, maximum delivery viscosity also decreases rapidly. If catheter ID is reduced to 0.025-0.027″ (common sizing), viscosity must be <1000 cP for delivery at 1 mL per minute. As catheter size is further reduced to 0.018″ ID (small peripheral vascular catheter), a viscosity of 236 cP would be required to maintain this flow rate. In small neurovascular microcatheters (≤0.013″), a viscosity of <70 cP would be required for an embolic deliverable at 1 mL per minute. As the figures show, viscosity requirements vary greatly across catheter size and desired flow rate. Given these catheter limitations, precise control of viscosity is an essential characteristic of a liquid embolic technology platform. Higher viscosity solutions are appropriate for use in large catheters, while viscosity must be decreased dramatically for use in small microcatheters.

Finally, the injectable compositions can be readily and easily prepared as needed. As will be discussed below, the injectable compositions can be prepared in a number of different ways depending upon the application of the compositions.

Each component used to produce the injectable compositions described herein as well as methods for making the injectable compositions is provided below.

### Transient Contrast Agent

The injectable compositions described herein include one or more transient contrast agents, where the contrast agent readily diffuses out of the solid produced in situ upon administration to the subject, providing temporary contrast.

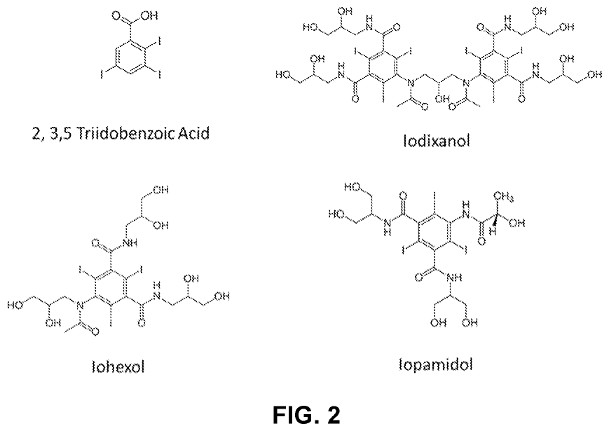

In one aspect, the transient contrast agent is a non-ionic compound. In another aspect, the transient contrast agent is water-soluble. In one aspect, the transient contrast agent is an iodinated organic compound, where one or more iodine atoms are covalently bonded to the organic compound. Iodinated organic contrast agents are a class of iodine-containing organic compounds. This set of compounds are derivatives of 2,3,5-triidobenzoic acid to produce different commercially available compounds, such as iopamidol, iodixanol, iohexol, iopromide, iobtiridol, iomeprol, iopentol, iopamiron, ioxilan, iotrolan, iotrol and ioversol, iopanoate, diatrizoic acid, iothalamate, and ioxaglate, various side chains are added to the parent compound. These sidechains modify the solubility, toxicity, and osmolality of the compound. Iodixanol is a dimer of the parent compound, producing a molecule with 6 iodine atoms. Structures for these compounds and the parent compound 2, 3, 5-triidobenzoic acid are shown in FIG. 2. In another aspect, the iodinated organic compound is an iodinated oil such as, for example, ethiodized poppyseed oil (Lipiodol).

The concentration of the transient contrast agent in the injectable compositions can vary depending upon the application. In one aspect, the concentration of the transient contrast agent in the injectable composition is from 10 mgI/mL to 1,000 mgI/mL, or is 10 mgI/mL, 25 mgI/mL, 50 mgI/mL, 75 mgI/mL, 100 mgI/mL, 125 mgI/mL, 150 mgI/mL, 175 mgI/mL, 200 mgI/mL, 225 mgI/mL, 250 mgI/mL, 275 mgI/mL, 300 mgI/mL, 325 mgI/mL, 350 mgI/mL, 375 mgI/mL, 400 mgI/mL, 425 mgI/mL, 450 mgI/mL, 475 mgI/mL, 500 mgI/mL, 525 mgI/mL, 550 mgI/mL, 575 mgI/mL, 600 mgI/mL, 625 mgI/mL, 650 mgI/mL, 675 mgI/mL, 700 mgI/mL, 725 mgI/mL, 750 mgI/mL, 775 mgI/mL, 800 mgI/mL, 825 mgI/mL, 850 mgI/mL, 875 mgI/mL, 900 mgI/mL, 925 mgI/mL, 950 mgI/mL, 975 mgI/mL, or 1,000 mgI/mL, 100 mgI/mL, 100 mgI/mL, 100 mgI/mL, 100 mgI/mL, 100 mgI/mL, 100 mgI/mL, where any value can be a lower and upper end-point of a range (e.g., 400 mgI/mL to 600 mgI/mL, etc.).

In one aspect, the majority of the transient contrast agent that diffuses from the solid is such that the transient contrast agent cannot be detected by imaging techniques such as, for example, fluoroscopy or CT. In one aspect, up to 70%, up to 80%, up to 90%, up to 95%, or up to 100% of the transient contrast agent diffuses out of the solid from 5 minutes to 48 hours once the solid is produced in situ, or 5 minutes, 10 minutes, 30 minutes, 1 hour, 2 hours, 3 hours, 4 hours, 5 hours, 6 hours, 7 hours, 8 hours, 9 hours, 10 hours, 11 hours, 12 hours, 18 hours, 24 hours, 30 hours, 36 hours, 42 hours, or 48 hours, 2 days, 5 days, 10 days, 15 days, 20 days, 25 days, or 30 days, where any value can be a lower and upper end-point of a range (e.g., 1 hour to 3 hours, etc.).

### Polycationic Salts

The polycationic salt is compound having a plurality of cationic groups and pharmaceutically-acceptable counterions, where there is a 1:1 stoichiometric ratio of the cationic groups to anionic counterions. In one aspect, the polycationic salt is a polymer having a polymer backbone with a plurality of cationic groups and pharmaceutically-acceptable anionic counterions. The cationic groups can be pendant to the polymer backbone and/or incorporated within the polymer backbone.

In one aspect, the polycationic polyelectrolyte is derived by dissolving a polycationic salt in water. In one aspect, the polycationic salt is a polycationic hydrochloride salt, wherein upon mixing with water produces the polycationic polyelectrolyte and chloride ions. In another aspect, the polycationic salts described herein can be produced by combining a polymer with a plurality of basic groups (e.g., amino groups) with an acid to produce the corresponding cationic groups. In various aspects, acids which may be employed to form pharmaceutically acceptable polycationic salts include inorganic acids as hydrochloric acid, acetic acid, or other monovalent carboxylic acids.

Also, basic nitrogen-containing groups can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

In other aspects, when the polycationic salt is a polymer, the polycationic salt can be produced by the polymerization of one or more monomers, where the monomers possess one or more cationic groups with corresponding counterion. Non-limiting procedures for making the polycationic salts using this approach are provided in the Examples. In one aspect, once the polycation has been prepared, excess ions can be removed from the polycation by filtration or dialysis prior to drying (e.g., lyophilization) to produce the polycationic salt with stoichiometric amounts of anionic counterions relative to the number of cationic groups.

In one aspect, the counterion of the polycationic salt is a monovalent ion such as, for example, chloride, pyruvate, acetate, tosylate, benzenesulfonate, benzoate, lactate, salicylate, glucuronate, galacturonate, nitrite, mesylate, trifluoroacetate, nitrate, gluconate, glycolate, formate, or any combination thereof. In one aspect, the counterion of the polycationic salt is a multivalent ion such as, for example, sulfate or phosphate.

In one aspect, the polycationic salt is a pharmaceutically-acceptable salt of a polyamine. The amino groups of the polyamine can be branched or part of the polymer backbone. In one aspect, the polyamine comprises two or more pendant amino groups, wherein the amino group comprises a primary amino group, a secondary amino group, tertiary amino group, a quaternary amine, an alkylamino group, a heteroaryl group, a guanidinyl group, an imidazolyl, or an aromatic group substituted with one or more amino groups.

In one aspect, the pharmaceutically-acceptable salt of the polyamine can include an aryl group having one or more amino groups directly or indirectly attached to the aromatic group. Alternatively, the amino group can be incorporated in the aromatic ring. For example, the aromatic amino group is a pyrrole, an isopyrrole, a pyrazole, imidazole, a triazole, or an indole. In another aspect, the aromatic amino group includes the isoimidazole group present in histidine. In another aspect, the biodegradable polyamine can be gelatin modified with ethylenediamine.

The amino group of the polyamine can be protonated at a pH of from about 6 to about 9 (e.g., physiological pH) to produce cationic ammonium groups with a pharmaceutically-acceptable counterion.

In general, the polyamine salt is a polymer with a large excess of positive charges relative to negative charge at or near physiological pH. For example, the polycationic salt can have from 10 to 90 mole %, 10 to 80 mole %, 10 to 70 mole %, 10 to 60 mole %, 10 to 50 mole %, 10 to 40 mole %, 10 to 30 mole %, or 10 to 20 mole % protonated amino groups. In another aspect, all of the amino groups of the polyamine are protonated.

In one aspect, the polycationic salt can have a protonated residue of lysine, histidine, or arginine. For example, arginine has a guanidinyl group, where the guanidinyl group is a suitable amino group that can be converted to a cationic group useful herein.

In another aspect, the polyamine can be a biodegradable synthetic polymer or naturally-occurring polymer. The mechanism by which the polyamine can degrade will vary depending upon the polyamine that is used. In the case of natural polymers, they are biodegradable because there are enzymes that can hydrolyze the polymer chain. For example, proteases can hydrolyze natural proteins like gelatin. In the case of synthetic biodegradable polyamines, they also possess chemically labile bonds. For example, (3-aminoesters have hydrolyzable ester groups.

In one aspect, the polyamine includes a polysaccharide, a protein, peptide, or a synthetic polyamine. Polysaccharides bearing two or more amino groups can be used herein. In one aspect, the polysaccharide is a natural polysaccharide such as chitosan or chemically modified chitosan. Similarly, the protein can be a synthetic or naturally-occurring compound. In another aspect, the polyamine is a synthetic polyamine such as poly(β-aminoesters), polyester amines, poly(disulfide amines), mixed poly(ester and amide amines), and peptide crosslinked polyamines.

In one aspect, the pharmaceutically-acceptable salt of the polyamine can be an amine-modified natural polymer. For example, the amine-modified natural polymer can be gelatin modified with one or more alkylamino groups, heteroaryl groups, or an aromatic group substituted with one or more amino groups. Examples of alkylamino groups are depicted in Formulae IV-VI

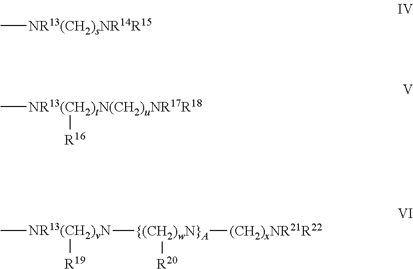

wherein R¹³-R²² are, independently, hydrogen, an alkyl group, or a nitrogen containing substituent;
s, t, u, v, w, and x are an integer from 1 to 10; and
A is an integer from 1 to 50,
where the alkylamino group is covalently attached to the natural polymer. In one aspect, if the natural polymer has a carboxyl group (e.g., acid or ester), the carboxyl group can be reacted with an alkyldiamino compound to produce an amide bond and incorporate the alkylamino group into the polymer. Thus, referring to formulae IV-VI, the amino group NR¹³ is covalently attached to the carbonyl group of the natural polymer.

As shown in formula IV-VI, the number of amino groups can vary. In one aspect, the alkylamino group is

—NHCH₂NH₂, —NHCH₂CH₂NH₂, —NHCH₂CH₂CH₂NH₂, -NHCH₂CH₂CH₂CH₂NH₂,
—NHCH₂CH₂CH₂CH₂CH₂NH₂,
—NHCH₂NHCH₂CH₂CH₂NH₂,
—NHCH₂CH₂NHCH₂CH₂CH₂NH₂,
—NHCH₂CH₂CH₂NHCH₂CH₂CH₂CH₂NHCH₂CH₂CH₂NH₂,
—NHCH₂CH₂NHCH₂CH₂CH₂CH₂NH₂,
—NHCH₂CH₂NHCH₂CH₂CH₂NHCH₂CH₂CH₂NH₂, or
—NHCH₂CH₂NH(CH₂CH₂NH)dCH₂CH₂NH₂, where d is from 0 to 50.

In one aspect, the pharmaceutically-acceptable salt of the amine-modified natural polymer can include an aryl group having one or more amino groups directly or indirectly attached to the aromatic group. Alternatively, the amino group can be incorporated in the aromatic ring. For example, the aromatic amino group is a pyrrole, an isopyrrole, a pyrazole, imidazole, a triazole, or an indole. In another aspect, the aromatic amino group includes the isoimidazole group present in histidine. In another aspect, the biodegradable polyamine can be gelatin modified with ethylenediamine.

In other aspects, the polycationic salt can be a dendrimer. The dendrimer can be a branched polymer, a multi-armed polymer, a star polymer, and the like. In one aspect, the dendrimer is a polyalkylimine dendrimer, a mixed amino/ether dendrimer, a mixed amino/amide dendrimer, or an amino acid dendrimer. In another aspect, the dendrimer is poly(amidoamine), or PAMAM. In one aspect, the dendrimer has 3 to 20 arms, wherein each arm comprises an amino group.

In one aspect, the polycationic salt includes a polyacrylate having one or more pendant protonated amino groups. For example, the backbone of the polycationic salt can be derived from the polymerization of acrylate monomers including, but not limited to, acrylates, methacrylates, acrylamides, methacrylamides, and the like. In one aspect, the polycationic salt backbone is derived from polyacrylamide. In other aspects, the polycationic salt is a random co-polymer. In other aspects, the polycationic salt is a block copolymer, where segments or portions of the co-polymer possess cationic groups or neutral groups depending upon the selection of the monomers and method used to produce the co-polymer.

In another aspect, the polycationic salt is a pharmaceutically-acceptable salt of a protamine. Protamines are polycationic, arginine-rich proteins that play a role in condensation of chromatin into the sperm head during spermatogenesis. As by-products of the fishing industry, commercially available protamines, purified from fish sperm, are readily available in large quantity and are relatively inexpensive. A non-limiting example of a protamine useful herein is salmine. In another aspect, the protamine is clupein.

In one aspect, the polycationic salts is a polymer with a plurality of guanidinyl groups. In one aspect, the guanidinyl groups are pendant to the polymer backbone. The number of guanidinyl groups present on the polycation ultimately determines the charge density of the polycation. In one aspect, the guanidinyl group can be derived from a residue of arginine attached to a polymer backbone.

The polyguanidinyl polymer can be a homopolymer or copolymer having a plurality of guanidinyl groups. In one aspect, the polyguanidinyl copolymer is a synthetic compound prepared by the free radical polymerization between a monomer such as an acrylate, a methacrylate, an acrylamide, a methacrylamide, or any combination thereof, and a guanidinyl monomer of formula I

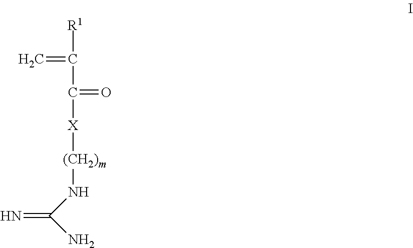

wherein R¹ is a hydrogen or an alkyl group, X is oxygen or NR⁵ , where R⁵ is a hydrogen or an alkyl group, and m is from 1 to 10, or the pharmaceutically acceptable salt thereof. In one aspect, when the neutral compound of formula I is used to produce the polymer, the resulting polymer can be subsequently reacted with an acid such as, for example, hydrochloric acid or ammonium chloride, to produce the polycationic salt.

In one aspect, in the compound of formula I, R¹ is methyl, X is NH, and m is 3. In another aspect, the monomer is methacrylamide, methacrylamide, N-(2-hydroxypropyl)methacrylamide (HPMA), N-[3-(N′-dicarboxymethyl)aminopropyl]methacrylamide (DAMA), N-(3-aminopropyl)methacrylamide, N-(1,3-dihydroxypropan-2-yl) methacrylamide, N-isopropylmethacrylamide, N-hydroxyethylacrylamide (HEMA), or any combination thereof.

In a further aspect, the mole ratio of the guanidinyl monomer of formula Ito the monomer is from 1:20 to 20:1, or is 1:20, 1:19, 1:18, 1:17, 1:16, 1:15, 1:14, 1:13, 1:12, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1,or 20:1, where any ratio can be a lower and upper end-point of a range (e.g., 2:1 to 5:1, etc.). In one aspect, the mole ratio of the guanidinyl monomer of formula Ito the monomer is from 3:1 to 4:1. In another aspect, the polyguanidinyl polymer is a homopolymer derived from the guanidinyl monomer of formula I.

The polyguanidinyl copolymer can be synthesized using polymerization techniques known in the literature such as, for example, RAFT polymerization (i.e., reversible addition-fragmentation chain-transfer polymerization) or other methods such as free radical polymerization. In one aspect, the polymerization reaction can be carried out in an aqueous environment. As discussed above, the polyguanidinyl copolymer can be prepared initially as a neutral polymer followed by treatment with an acid to produce the pharmaceutically-acceptable salt.

In another aspect, multiple copolymers with controlled M_{w} and narrow polydispersity indices (PDIs) can be synthesized by RAFT polymerization. In one aspect, the pharmaceutically-acceptable salt of the polyguanidinyl copolymer has an average molecular weight (M_{w}) from about 1 kDa to about 100 kDa, or can be about 1, 2, 3, 4, 5, 10, 15, 20, 25, 30, 35, 40, 45, 50, 55, 60, 65, 70, 75, 80, 85, 90, 95, or 100 kDa, where any value can be a lower and upper end-point of a range (e.g., 10 to 25 kDa, etc.).

In another aspect, the pharmaceutically-acceptable salt of the polyguanidinyl copolymer is a multimodal polyguanidinyl copolymer. The term “multimodal polyguanidinyl copolymer” is a polyguanidinyl copolymer with a molecular mass distribution curve being the sum of at least two or more molecular mass unimodal distribution curves. In one aspect, the polyguanidinyl copolymer has a multimodal distribution of polyguanidinyl copolymer molecular mass with modes between 5 and 100 kDa, or can be about 5, 10, 15, 20, 25, 30, 35, 40, 45, 50, 55, 60, 65, 70, 75, 80, 85, 90, 95, or 100 kDa, where any value can be a lower and upper end-point of a range (e.g., 10 to 30 kDa, etc.).

In another aspect, the number of guanidinyl side groups in the pharmaceutically-acceptable salt of the polyguanidinyl copolymer can vary from about 10 to about 100 mol % of the. total polymer sidechains, or can be about 10, 15, 20, 25, 30, 35, 40, 45, 50, 55, 60, 65, 70, 75, 80, 85, 90, 95, or 100 mol %, where any value can be a lower and upper end-point of a range (e.g., 60 to 90 mol %, etc.). In one aspect, the guanidinyl side groups are from about 70 to about 80 mol % of the polyguanidinyl copolymer. Conversely, comonomer concentration can vary from about 50 to about 0 mol %, or can be about 50, 45, 40, 35, 30, 25, 20, 15, 10, 5, or 0 mol %, where any value can be a lower and upper end-point of a range (e.g., 10 to 40 mol %, etc.). In one aspect, the Mₙ, PDI, and structures of the copolymers can be verified by size exclusion chromatography (SEC), ₁H NMR, and ¹³C NMR or other common techniques. Exemplary procedures for preparing and characterizing copolymers useful herein are provided in the Examples below.

The concentration of the of the polycationic salt in the injectable compositions described herein can vary depending upon the application of the composition. In one aspect, the concentration of the of the polycationic salt used to produce the injectable compositions described herein is from 100 mg/mL to 1,000 mg/mL, or 100 mg/mL, 100 mg/mL, 150 mg/mL, 200 mg/mL, 250 mg/mL, 300 mg/mL, 350 mg/mL, 400 mg/mL, 450 mg/mL, 500 mg/mL, 550 mg/mL, 600 mg/mL, 650 mg/mL, 700 mg/mL, 750 mg/mL, 800 mg/mL, 850 mg/mL, 900 mg/mL, 950 mg/mL, 1,000 mg/mL, where any value can be a lower and upper end-point of a range (e.g., 200 mg/mL to 500 mg/mL, etc.).

### Polyanionic Salts

The polyanionic salt is a compound with a plurality of anionic groups and pharmaceutically-acceptable cationic counterions, where there is a 1:1 stoichiometric ratio of the anionic groups to cationic counterions.

In one aspect, the polyanionic polyelectrolyte is derived by dissolving a polyanionic salt in water. In one aspect, the polyanionic salts described herein can be produced by adjusting the pH of a solution of a compound with a plurality of acidic groups (e.g., carboxylic acid groups) with the addition of a base to produce the corresponding anionic groups. In various aspects, bases which may be employed to form pharmaceutically acceptable polyanionic salts include alkali metal hydroxides, carbonates, acetate, etc. In one aspect, once the polyanion has been prepared, excess ions can be removed from the polyanion by filtration or dialysis prior to drying (e.g., lyophilization) to produce the polyanionic salt with stoichiometric amounts of cationic counterions relative to the number of anionic groups.

In one aspect, the cationic counterions of the polyanionic salt are monovalent cations such as, for example, sodium, potassium or ammonium ions. In another aspect, the counterions of the polyanionic salt are multivalent ion such as, for example, calcium, magnesium ions, or mixtures thereof.

In one aspect, the polyanionic salt is composed of a polymer backbone with a plurality of anionic groups and pharmaceutically-acceptable cationic counterions. The anionic groups can be pendant to the polymer backbone and/or incorporated within the polymer backbone. In certain aspects, (e.g., biomedical applications), the polyanionic salt is any biocompatible polymer possessing anionic groups.

In one aspect, the polyanionic salt can be a pharmaceutically-acceptable salt of a synthetic polymer or naturally-occurring polymer. Examples of naturally-occurring polyanions include glycosaminoglycans such as chondroitin sulfate, heparin, heparin sulfate, dermatan sulfate, keratin sulfate, and hyaluronic acid. In other aspects, proteins having a net negative charge at neutral pH or proteins with a low pI can be used as naturally-occurring polyanions described herein. The anionic groups can be pendant to the polymer backbone and/or incorporated in the polymer backbone.

When the polyanionic salt is a synthetic polymer, it is generally any polymer possessing anionic groups or groups that can be ionized to anionic groups. Examples of groups that can be converted to anionic groups include, but are not limited to, carboxylate, sulfonate, boronate, sulfate, borate, phosphonate, or phosphate.

In one aspect, the polyanionic salt is a polyphosphate. In another aspect, the polyanionic salt is a polyphosphate compound having from 5 to 90 mole % phosphate groups. In another aspect, the polyanionic salt has from 10 to 1,000 phosphate groups, or 10, 50, 100, 150, 200, 250, 300, 350, 400, 450, 500, 550, 600, 650, 700, 750, 800, 850, 900, 950, or 1,000 phosphate groups, where any value can be a lower and upper end-point of a range (e.g., 100 to 300, etc.).

In one aspect, the polyphosphate can be a naturally-occurring compound such as, for example, DNA, RNA, or highly phosphorylated proteins like phosvitin (an egg protein), dentin (a natural tooth phosphoprotein), casein (a phosphorylated milk protein), or bone proteins (e.g. osteopontin).

In another aspect, the polyanionic salt can be a synthetic polypeptide made by polymerizing the amino acid serine and then chemically or enzymatically phosphorylating the polypeptide. In another aspect, the polyanionic salt can be produced by the polymerization of phosphoserine. In one aspect, the polyphosphate can be produced by chemically or enzymatically phosphorylating a protein (e.g., natural serine- or threonine-rich proteins). In a further aspect, the polyphosphate can be produced by chemically phosphorylating a polyalcohol including, but not limited to, polysaccharides such as cellulose or dextran. The polyanionic polymers can subsequently be converted to pharmaceutically-acceptable salts.

In another aspect, the polyphosphate can be a synthetic compound. For example, the polyphosphate can be a polymer with pendant phosphate groups attached to the polymer backbone and/or present in the polymer backbone. (e.g., a phosphodiester backbone).

In one aspect, the polyanionic salt includes a polyacrylate having one or more pendant phosphate groups. For example, the polyanionic salt can be derived from the polymerization of acrylate monomers including, but not limited to, acrylates, methacrylates, acrylamides, methacrylamides, and the like. In other aspects, the polyanionic salt is a block co-polymer, where segments or portions of the co-polymer possess anionic groups and neutral groups depending upon the selection of the monomers used to produce the co-polymer. In one aspect, the anionic group can be a plurality of carboxylate, sulfate, sulfonate, borate, boronate, phosphonate, or phosphate groups.

In one aspect, the polyanionic salt is a polymer having a plurality of fragments of formula XI

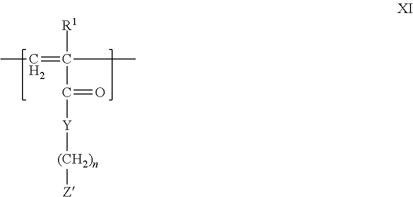

wherein R⁴ is hydrogen or an alkyl group;
n is from 1 to 10;
Y is oxygen, sulfur, or NR³⁰ , wherein R³⁰ is hydrogen, an alkyl group, or an aryl group;
Z′ is a pharmaceutically-acceptable salt of an anionic group.

In one aspect, Z′ in formula XI is carboxylate, sulfate, sulfonate, borate, boronate, a substituted or unsubstituted phosphate, or a phosphonate. In another aspect, Z′ in formula XI is sulfate, sulfonate, borate, boronate, a substituted or unsubstituted phosphate, or a phosphonate, and n in formulae XI is 2.

In one aspect, the polyanionic salt can be an inorganic polyphosphate including a cyclic inorganic polyphosphate having the formula (PₙO₃ₙ)ⁿ⁻, a linear inorganic polyphosphate having the formula (PₙO₃ₙ₊₁)ⁿ⁺²⁻, or a combination thereof. In one aspect, the polyanionic salt is an inorganic polyphosphate possessing a plurality of phosphate groups (e.g., NaPO₃)ₙ, where n is 10 to 1,000 or 10, 50, 100, 150, 200, 250, 300, 350, 400, 450, 500, 550, 600, 650, 700, 750, 800, 850, 900, 950, or 1,000 phosphate groups, where any value can be a lower and upper end-point of a range (e.g., 100 to 300, etc.). Examples of inorganic phosphates include, but are not limited to, Graham salts, hexametaphosphate salts, and triphosphate salts. The counterions of these salts can be monovalent cations such as, for example, Na⁺, K⁺, NH₄⁺, or a combination thereof. In one aspect, the polyanionic salt is sodium hexametaphosphate.

In another aspect, the polyanionic salt is an organic polyphosphate. In one aspect, polymers with phosphodiester backbones connecting organic moieties (e.g., DNA or synthetic phosphodiesters) are organic polyphosphates useful herein.

In another aspect, the polyanionic salt is a pharmaceutically-acceptable salt of a phosphorylated sugar. The sugar can be a hexose or pentose sugar. Additionally, the sugar can be partially or fully phosphorylated. In one aspect, the phosphorylated sugar is inositol hexaphosphate (IP6).

The concentration of the of the polyanionic salt in the injectable compositions described herein can vary depending upon the application of the composition. In one aspect, the concentration of the of the polyanionic salt used to produce the injectable compositions described herein is from 100 mg/mL to 1,000 mg/mL, or 100 mg/mL, 100 mg/mL, 150 mg/mL, 200 mg/mL, 250 mg/mL, 300 mg/mL, 350 mg/mL, 400 mg/mL, 450 mg/mL, 500 mg/mL, 550 mg/mL, 600 mg/mL, 650 mg/mL, 700 mg/mL, 750 mg/mL, 800 mg/mL, 850 mg/mL, 900 mg/mL, 950 mg/mL, 1,000 mg/mL, where any value can be a lower and upper end-point of a range (e.g., 200 mg/mL to 500 mg/mL, etc.).

### Reinforcing Component

In another aspect, the injectable compositions described herein also include a reinforcing component. The term “reinforcing component” is defined herein as any component that enhances or modifies one or more mechanical or physical properties of the solids produced herein (e.g., cohesiveness, fracture toughness, elastic modulus, dimensional stability after curing, color, visibility etc.). The mode in which the reinforcing component can enhance the mechanical properties of the solid can vary and will depend on the selection of the components used to prepare the injectable composition and reinforcing component. Examples of reinforcing component useful herein are provided below.

In one aspect, the reinforcing component is a coil or fiber. In a further aspect, the coil or fiber can be platinum, plastic, nylon, another natural or synthetic fiber, a polymerizable monomer, a nanostructure, a micelle, a liposome, a water-insoluble filler, or any combination thereof. In one aspect, the coil or fiber is administered concurrently with the injectable composition. In another aspect, the coil or fiber is administered sequentially either before or after the injectable composition.

In other aspects, the reinforcing component can be a water-insoluble filler. The filler can have a variety of different sizes and shapes, ranging from particles (micro and nano) to fibrous materials. The selection of the filler can vary depending upon the application of the injectable composition.

The fillers useful herein can be composed of organic and/or inorganic materials. In one aspect, the nanostructures can be composed of organic materials like carbon or inorganic materials including, but not limited to, boron, molybdenum, tungsten, silicon, titanium, copper, bismuth, tungsten carbide, aluminum oxide, titanium dioxide, molybdenum disulphide, silicon carbide, titanium diboride, boron nitride, dysprosium oxide, iron (III) oxide-hydroxide, iron oxide, manganese oxide, titanium dioxide, boron carbide, aluminum nitride, or any combination thereof.

In one aspect, the filler comprises a metal oxide, a ceramic particle, or a water insoluble inorganic salt. Examples of fillers useful herein include those manufactured by SkySpring Nanomaterials, Inc., which is listed below.

### Metals and Non-Metal Elements

### Ag, 99.95%, 100 nm

### Ag, 99.95%, 20-30 nm

Ag, 99.95%, 20-30 nm, PVP coated

### Ag, 99.9%, 50-60 nm

Ag, 99.99%, 30-50 nm, oleic acid coated
Ag, 99.99%, 15 nm, 10 wt %, self-dispersible
Ag, 99.99%, 15 nm, 25wt %, self-dispersible

### Al, 99.9%, 18 nm

### Al, 99.9%, 40-60 nm

### Al, 99.9%, 60-80 nm

Al, 99.9%, 40-60 nm, low oxygen

### Au, 99.9%, 100 nm

Au, 99.99%, 15 nm, 10 wt %, self-dispersible

### B, 99.9999%

### B, 99.999%

### B, 99.99%

### B, 99.9%

### B, 99.9%, 80 nm

### Diamond, 95%, 3-4 nm

### Diamond, 93%, 3-4 nm

### Diamond, 55-75%, 4-15 nm

### Graphite, 93%, 3-4 nm

### Super Activated Carbon, 100 nm

### Co, 99.8%, 25-30 nm

### Cr, 99.9%, 60-80 nm

### Cu, 99.5%, 300 nm

### Cu, 99.5%, 500 nm

### Cu, 99.9%, 25 nm

### Cu, 99.9%, 40-60 nm

### Cu, 99.9%, 60-80 nm

Cu, 5-7 nm, dispersion, oil soluble

### Fe, 99.9%, 20 nm

### Fe, 99.9%, 40-60 nm

### Fe, 99.9%, 60-80 nm

Carbonyl-Fe, micro-sized

### Mo, 99.9%, 60-80 nm

### Mo, 99.9%, 0.5-0.8 lam

Ni, 99.9%, 500 nm (adjustable)

### Ni, 99.9%, 20 nm

Ni coated with carbon, 99.9%, 20 nm

### Ni, 99.9%, 40-60 nm

### Ni, 99.9%, 60-80 nm

### Carbonyl-Ni, 2-3 nm

### Carbonyl-Ni, 4-7 nm

### Carbonyl-Ni—Al (Ni Shell, Al Core)

### Carbonyl-Ni—Fe Alloy

Pt, 99.95%, 5 nm, 10 wt %, self-dispersible

### Si, Cubic, 99%, 50 nm

Si, Polycrystalline, 99.99995%, lumps

### Sn, 99.9%, <100 nm

### Ta, 99.9%, 60-80 nm

### Ti, 99.9%, 40-60 nm

### Ti, 99.9%, 60-80 nm

### W, 99.9%, 40-60 nm

### W, 99.9%, 80-100 nm

### Zn, 99.9%, 40-60 nm

### Zn, 99.9%, 80-100 nm

### Metal Oxides

AlOOH, 10-20nm, 99.99%
Al₂O₃ alpha, 98+%, 40 nm
Al₂O₃ alpha, 99.999%, 0.5-10 μm
Al₂O₃ alpha, 99.99%, 50 nm
Al₂O₃ alpha, 99.99%, 0.3-0.8 μm
Al₂O₃ alpha, 99.99%, 0.8-1.5 μm
Al₂O₃ alpha, 99.99%, 1.5-3.5 μm
Al₂O₃ alpha, 99.99%, 3.5-15 μm
Al₂O₃ gamma, 99.9%, 5 nm
Al₂O₃ gamma, 99.99%, 20 nm
Al₂O₃ gamma, 99.99%, 0.4-1.5 μm
Al₂O₃ gamma, 99.99%, 3-10 μm
Al₂O₃ gamma, Extrudate
Al₂O₃ gamma, Extrudate
Al(OH)₃, 99.99%, 30-100 nm Al(OH)₃, 99.99%, 2-10 μm

Aluminium Iso-Propoxide (AIP), C₉H₂₁O₃Al, 99.9%

### AlN, 99%, 40 nm

### BaTiO₃, 99.9%, 100 nm

### BBr₃, 99.9%

B₂O₃, 99.5%, 80 nm

### BN, 99.99%, 3-4 μm

### BN, 99.9%, 3-4 μm

### B₄C, 99%, 50 nm

Bi₂O₃, 99.9%, <200 nm

### CaCO₃, 97.5%, 15-40 nm

### CaCO₃, 15-40 nm

Ca₃(PO₄)₂, 20-40 nm
Ca₃(PO₄)₆(OH)₂, 98.5%, 40 nm

### CeO₂, 99.9%, 10-30 nm

### CoO, <100 nm

Co₂O₃, <100 nm
Co₃O₄, 50 nm

### CuO, 99+%, 40 nm

Er₂O₃, 99.9%, 40-50 nm
Fe₂O₃ alpha, 99%, 20-40 nm
Fe₂O₃ gamma, 99%, 20-40 nm
Fe₃O₄, 98+%, 20-30 nm
Fe₃O₄, 98+%, 10-20 nm
Gd₂O₃, 99.9%<100 nm

### HfO₂, 99.9%, 100 nm

In₂O₃: SnO₂=90:10, 20-70 nm
In₂O₃, 99.99%, 20-70 nm

### In(OH)₃, 99.99%, 20-70 nm

### LaB₆, 99.0%, 50-80 nm

La₂O₃, 99.99%, 100 nm

### LiFePO₄, 40 nm

### MgO, 99.9%, 10-30 nm

### MgO, 99%, 20 nm

### MgO, 99.9%, 10-30 nm

### Mg(OH)₂, 99.8%, 50 nm

Mn₂O₃, 98+%, 40-60 nm

### MoCl₅, 99.0%

Nd₂O₃, 99.9%, <100 nm

### NiO, <100 nm

Ni₂O₃, <100 nm
Sb₂O₃, 99.9%, 150 nm

### SiO₂, 99.9%, 20-60 nm

SiO₂, 99%, 10-30 nm, treated with Silane Coupling Agents
SiO₂, 99%, 10-30 nm, treated with Hexamethyldisilazane
SiO₂, 99%, 10-30 nm, treated with Titanium Ester
SiO₂, 99%, 10-30 nm, treated with Silanes
SiO₂, 10-20 nm, modified with amino group, dispersible
SiO₂, 10-20 nm, modified with epoxy group, dispersible
SiO₂, 10-20 nm, modified with double bond, dispersible
SiO₂, 10-20 nm, surface modified with double layer, dispersible
SiO₂, 10-20 nm, surface modified, super-hydrophobic & oleophilic, dispersible
SiO₂, 99.8%, 5-15 nm, surface modified, hydrophobic & oleophilic, dispersible
SiO₂, 99.8%, 10-25 nm, surface modified, super-hydrophobic, dispersible
SiC, beta, 99%, 40 nm
SiC, beta, whisker, 99.9%
Si₃N₄, amorphous, 99%, 20 nm
Si₃N₄ alpha, 97.5-99%, fiber, 100 nm×800 nm

### SnO₂, 99.9%, 50-70 nm

ATO, SnO₂: Sb₂O₃=90:10, 40 nm
TiO₂ anatase, 99.5%, 5-10 nm

### TiO₂ Rutile, 99.5%, 10-30 nm

TiO₂ Rutile, 99%, 20-40 nm, coated with SiO₂, highly hydrophobic
TiO₂ Rutile, 99%, 20-40 nm, coated with SiO₂/Al₂O₃
TiO₂ Rutile, 99%, 20-40 nm, coated with Al₂O₃, hydrophilic
TiO₂ Rutile, 99%, 20-40 nm, coated with SiO₂/Al₂O₃/Stearic Acid
TiO₂ Rutile, 99%, 20-40 nm, coated with Silicone Oil, hydrophobic

### TiC, 99%, 40 nm

### TiN, 97+%, 20 nm

### WO₃, 99.5%, <100 nm

### WS₂, 99.9%, 0.8 μm

### WCl₆, 99.0%

Y₂O₃, 99.995%, 30-50 nm

### ZnO, 99.8%, 10-30 nm

ZnO, 99%, 10-30 nm, treated with silane coupling agents
ZnO, 99%, 10-30 nm, treated with stearic acid
ZnO, 99%, 10-30 nm, treated with silicone oil

### ZnO, 99.8%, 200 nm

### ZrO₂, 99.9%, 100 nm

### ZrO₂, 99.9%, 20-30 nm

### ZrO₂-3Y, 99.9%, 0.3-0.5 μm

### ZrO₂-3Y, 25 nm

### ZrO₂-5Y, 20-30 nm

### ZrO₂-8Y, 99.9%, 0.3-0.5 μm

### ZrO₂-8Y, 20 nm

### ZrC, 97+%, 60 nm

In one aspect, the filler is nanosilica. Nanosilica is commercially available from multiple sources in a broad size range. For example, aqueous Nexsil colloidal silica is available in diameters from 6-85 nm from Nyacol Nanotechnologies, Inc.

Amino-modified nanosilica is also commercially available, from Sigma Aldrich for example, but in a narrower range of diameters than unmodified silica.

In another aspect, the filler can be composed of calcium phosphate. In one aspect, the filler can be hydroxyapatite, which has the formula Ca₅(PO₄)₃OH. In another aspect, the filler can be a substituted hydroxyapatite. A substituted hydroxyapatite is hydroxyapatite with one or more atoms substituted with another atom. The substituted hydroxyapatite is depicted by the formula M₅X₃Y, where M is Ca, Mg, Na; X is PO₄ or CO₃; and Y is OH, F, Cl, or CO₃. Minor impurities in the hydroxyapatite structure may also be present from the following ions: Zn, Sr, Al, Pb, Ba. In another aspect, the calcium phosphate comprises a calcium orthophosphate. Examples of calcium orthophosphates include, but are not limited to, monocalcium phosphate anhydrate, monocalcium phosphate monohydrate, dicalcium phosphate dihydrate, dicalcium phosphate anhydrous, octacalcium phosphate, beta tricalcium phosphate, alpha tricalcium phosphate, super alpha tricalcium phosphate, tetracalcium phosphate, amorphous tricalcium phosphate, or any combination thereof. In other aspects, the calcium phosphate can also include calcium-deficient hydroxyapatite, which can preferentially adsorb bone matrix proteins.

In certain aspects, the filler can be functionalized with one or more amino or activated ester groups. In this aspect, the filler can be covalently attached to the polycation or polyanion. For example, aminated silica can be reacted with the polyanion possessing activated ester groups to form new covalent bonds.

### Bioactive Agents

The injectable compositions described herein can include one or more bioactive agents. In one aspect, the bioactive agent is an antibiotic, a pain reliever, an immune modulator, a growth factor, an enzyme inhibitor, a hormone, a messenger molecule, a cell signaling molecule, a receptor agonist, an oncolytic virus, a chemotherapy agent, an anti-angiogenic agent, a receptor antagonist, a nucleic acid, or any combination thereof.

In one aspect, the bioactive agent can be a nucleic acid. The nucleic acid can be an oligonucleotide, deoxyribonucleic acid (DNA), ribonucleic acid (RNA) including mRNA, or peptide nucleic acid (PNA). The nucleic acid of interest can be a nucleic acid from any source, such as a nucleic acid obtained from cells in which it occurs in nature, recombinantly produced nucleic acid, or chemically synthesized nucleic acid, or chemically modified nucleic acids. For example, the nucleic acid can be cDNA or genomic DNA or DNA synthesized to have the nucleotide sequence corresponding to that of naturally-occurring DNA. The nucleic acid can also be a mutated or altered form of nucleic acid (e.g., DNA that differs from a naturally occurring DNA by an alteration, deletion, substitution or addition of at least one nucleic acid residue) or nucleic acid that does not occur in nature.

In other aspects, the bioactive agent is used in bone treatment applications. For example, the bioactive agent can be bone morphogenetic proteins (BMPs) and prostaglandins. When the bioactive agent is used to treat osteoporosis, bioactive agents known in the art such as, for example, bisphonates, can be delivered locally to the subject by the injectable compositions and solids produced therefrom.

In certain aspects, the filler used to produce the injectable composition can also possess bioactive properties. For example, when the filler is a silver particle, the particle can also behave as an anti-microbial agent. The rate of release can be controlled by the selection of the materials used to prepare the injectable composition, as well as the charge of the bioactive agent if the agent has ionizable groups. Thus, in this aspect, the solid produced from the injectable composition can perform as a localized controlled drug release depot. It may be possible to simultaneously fix tissue and bones as well as deliver bioactive agents to provide greater patient comfort, accelerate bone healing, and/or prevent infections.

In one aspect, the bioactive agent is an FDA-approved anti-angiogenic agent. In one aspect, the anti-angiogenic agent is a tyrosine kinase inhibitor (TM). Not wishing to be bound by theory, angiogenesis is, in large part, initiated and maintained by cell signaling through receptor tyrosine kinases (RTKs). In one aspect, RTKs include receptors for several angiogenesis promoters, including VEGF, which stimulates vascular permeability, proliferation, and migration of endothelial cells; PDGF, which recruits pericytes and smooth muscle cells that support the budding endothelium; and FGF, which stimulates proliferation of endothelial cells, smooth muscle cells, and fibroblasts. In one aspect, the anti-angiogenic agent is a TM such as sunitinib malate (SUN), pazopanib hydrochloride (PAZ), sorafenib tosylate (SOR), vandetanib (VAN), cabozantinib, or any combination thereof.

In another aspect, the bioactive agent can be humanized anti-VEGF and anti-VEGFR Fab′ fragments. In this aspect, electrostatic interactions can control release kinetics. In one aspect, the native charge of the Fab′ fragment is sufficient to interact with the polyelectrolyte components in the injectable composition. In another aspect, the native charge of the Fab′ fragment is insufficient to interact with the polyelectrolyte components in the injectable composition and the Fab′ fragment is modified to increase charge density by attaching a short polyelectrolyte to reactive sulfhydryl groups using maleamide conjugation chemistries.

In one aspect, the anti-angiogenic agent is an anti-VEGF antibody. In a still further aspect, the anti-VEGF antibody is bevacizumab or is a biosimilar anti-VEGF antibody, or is an anti-VEGF antibody derivative such as, for example, ranibizumab.

### Kits

Described herein are kits for making the injectable compositions. In one aspect, the kit includes (a) a composition comprising a mixture of at least one polycationic salt and at least one polyanionic salt, (b) a contrast agent, and (c) instructions for making the injectable composition. In another aspect, the kit includes (a) at least one polycationic salt, (b) at least one polyanionic salt, (c) a transient contrast agent, and (d) instructions for making the injectable composition.

The polycationic salt and polyanionic salt used herein can be stored as dry powders for extended periods of time. In one aspect, the kit can include dry powders of the polycationic salt and polyanionic salt as separate components in separate vials, or a mixture of the polycationic salt and polyanionic salt as a dry powder or solid in a single container. In other aspects, the kit can include aqueous solutions of the polycationic salt and polyanionic salt as separate components (e.g., in separate vials) or a mixture of the polycationic salt and polyanionic salt in water.

In one aspect, the kit can include the contrast as a dry powder or solid. In another aspect, the transient contrast agent can be in an aqueous solution or an oil.

The kits also include instructions for making the injectable compositions. As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can include one or multiple documents and are meant to include future updates.

The kits can also include additional components as described herein (e.g., reinforcing components, bioactive agents, etc.). In other aspects, the kits can include optional mechanical components such as, for example, syringes, microcatheters, and other devices for mixing and delivering the injectable compositions to a subject.

### Preparation of the Injectable Compositions

The preparation of the injectable compositions described herein can be performed using a number of techniques and procedures. Exemplary techniques for producing the injectable compositions are provided in the Examples. In one aspect, a powder composed of a mixture of the at least one polycationic salt and the at least one polyanionic salt are mixed with a composition comprising the transient contrast agent in water for a sufficient time to produce an injectable composition.

In another aspect, an aqueous solution composed of a mixture of the at least one polycationic salt and the at least one polyanionic salt are mixed with a composition comprising an oily transient contrast agent. In this aspect, the aqueous solution composed of the polyelectrolytes and the transient contrast agent in oil are mixed for a sufficient time to produce an emulsion.

In one aspect, one or more additional agents (e.g., reinforcing agent or bioactive agent) can be added after the injectable composition has been formed. In another aspect, the anti-angiogenic agent and the one or more additional agents (e.g., reinforcing agent or bioactive agent) can be added during the formation of the injectable composition.

In one aspect, the pH of the injectable composition is from 6 to 9, 6.5 to 8.5, 7 to 8, or 7 to 7.5. In another aspect, the pH of the composition is 7.2, which is the normal physiological pH in blood.

The injectable compositions described herein are stable solutions (i.e., a liquid composition of polyelectrolytes with no distinguishable separation into distinct phases).

Although the components used to produce the injectable composition can be used in dry powder form then subsequently mixed with water, the injectable compositions can be formulated as water-borne formulations and stored for future use. In certain aspects, one or more additional salts can be added to the injectable composition to prevent association of the polycationic polyelectrolytes and the polyanionic polyelectrolytes in the injectable composition. In one aspect, the salt is a monovalent salt. For example, sodium chloride can be added to the injectable composition to produce a stable composition as defined herein. The concentration of the monovalent salt can vary depending upon the molecular weight, concentration, and charge ratio of the polycationic and polyanionic salts. In other aspects, additional monovalent salt is not needed to produce the injectable compositions as stable solutions.

Depending upon the application site in the subject and delivery device dimensions, the viscosity of the of the injectable composition can be modified accordingly. This is an important feature with respect to medical applications such as, for example, transarterial microcatheter delivery, where different size microcatheters are needed for different applications. For example, modifying the concentration and/or molecular weight of the polycationic salt and/or the polyanionic salt can be used to modify the viscosity of the injectable composition.

In one aspect, the injectable composition has a viscosity of from 10 cp to 20,000 cp, or 10 cp, 25 cp, 50 cp, 75 cp, 100 cp, 125 cp, 150 cp, 200 cp, 225 cp, 250 cp, 275 cp, 300 cp, 325 cp, 350 cp, 375 cp, 400 cp, 425 cp, 450 cp, 475 cp, 500 cp, 1,000 cp, 1,500 cp, 2,000 cp, 2,500 cp, 3,000 cp, 3,500 cp, 4,000 cp, 4,500 cp, 5,000 cp, 5,500 cp, 6,000 cp, 6,500 cp, 7,000 cp, 7,500 cp, 8,000 cp, 8,500 cp, 9,000 cp, 9,500 cp, 10,000 cp, 11,000 cp, 12,000 cp, 13,000 cp, 14,000 cp, 15,000 cp, 10,000 cp, 16,000 cp, 17,000 cp, 18,000 cp, 19,000 cp, or 20,000 cp, where any value can be a lower and upper end-point of a range (e.g., 1,500 cp to 7,000 cp, etc.).

### Applications of the Injectable Compositions

The injectable compositions described herein have numerous benefits and biomedical applications. As discussed above, the injectable compositions are fluids that are readily injectable via a narrow-gauge device, catheter, needle, cannula, or tubing. The injectable compositions are water-borne eliminating the need for potentially toxic solvents.

The injectable compositions described herein are fluids at ion concentrations higher than the ion concentration of the application site in the subject, but insoluble solids at the ion concentration of the application site. When the injectable compositions are introduced into a subject at a lower ion concentration relative to the ion concentration of the injectable composition, the composition forms a porous solid in situ at the application site as the ion concentration in the injectable composition approaches the application site ion concentration. The solid that is subsequently produced has higher mechanical moduli than those of the initial fluid form of the injectable composition.

In one aspect, the injectable solution is delivered as pulses such that solid particles are periodically formed and released from the tip of the catheter within the subject. The in situ formed solid particles can be carried by the bloodstream to a distal location from the catheter tip to create a synthetic embolus.

In one aspect, the ion concentration of the injectable composition is the sum of the cationic and anionic counterions present in the composition. In another aspect, the ion concentration of the injectable composition is the sum of the cationic and anionic counterions present in the composition as well as additional ions that are added to the composition (e.g., the addition of NaCl to the composition). In one aspect, the composition has an ion concentration that is about 1.5 to about 20 times greater than the ion concentration in the subject, or about 1.5, 2, 3, 4, 5, 6, 7, 8, 9, 10, 11, 12, 13, 14, 15, 16, 17, 18, 19, or 20 times greater than the ion concentration in the subject, where any value can be a lower and upper end-point of a range (e.g., 2 times to 15 times). In another aspect, the ionic concentration in the composition is from 0.5 M to 2.0 M, or 0.5 M, 0.75 M, 1.0 M, 1.25 M, 1.5 M, 1.75 M, or 2.0 M, where any value can be a lower and upper end-point of a range (e.g., 0.75 M to 1.5 M).

The injectable compositions can form solids in situ under physiological conditions. The physiological sodium and chloride concentration is approximately 150 mM. Thus, when injectable compositions having an ion concentration greater than 150 mM are introduced to a subject (e.g., injected into a mammal), the injectable composition is converted to a porous solid at the site of application. Thus, the injectable compositions described herein have numerous medical and biological applications, which are described in detail below.

In one aspect, the injectable compositions and solids produced therefrom can be used to reduce or inhibit blood flow in a blood vessel of a subject. In this aspect, the solid produced from the injectable composition creates an artificial embolus within the blood vessel. Thus, the injectable compositions described herein can be used as synthetic embolic agents. In this aspect, the injectable composition is injected into the blood vessel followed by formation of the solid in order to partially or completely block the blood vessel. This method has numerous applications including the creation of an artificial embolism to inhibit blood flow to a tumor, aneurysm, varicose vein, an arteriovenous malformation, an open or bleeding wound, or other vascular trauma or defects. In other aspects, the injectable compositions can be administered in other areas in the subject including lymphatic vessels, ducts, airways, and other channels where it is desirable to form a solid in a medical application.

As discussed above, the injectable compositions can be used as synthetic embolic agents. However, in other aspects, the injectable composition described herein can include one or more additional embolic agents. Embolic agents commercially-available are microparticles used for embolization of blood vessels. The size and shape of the microparticles can vary. In one aspect, the microparticles can be composed of polymeric materials. An example of this is Bearin™nsPVA particles manufactured by Merit Medical Systems, Inc., which are composed of polyvinyl alcohol ranging in size from 45 μm to 1,180 μm. In another aspect, the embolic agent can be a microsphere composed of a polymeric material. Examples of such embolic agents include Embosphere® Microspheres, which are made from trisacryl cross-linked gelatin ranging in size from 40 μm to 1,200 μm; HepaSphere™ Microspheres (spherical, hydrophilic microspheres made from vinyl acetate and methyl acrylate) ranging in size from 30 μm to 200 μm; and QuadraSphere® Microspheres (spherical, hydrophilic microspheres made from vinyl acetate and methyl acrylate) ranging in size from 30 μm to 200 μm, all of which are manufactured by Merit Medical Systems, Inc. In another aspect, the microsphere can be impregnated with one or more metals that can be used as a contrast agent. An example of this is EmboGold® Microspheres manufactured by Merit Medical Systems, Inc., which are made from cross-linked trisacryl gelatin impregnated with 2% elemental gold ranging in size from 40 μm to 1,200 μm.

In another aspect, the injectable compositions described herein can be used in combination with one or more mechanical vascular devices such as, for example, embolic coils, fibers, and the like. In one aspect, the mechanical embolic is first administered to a blood vessel in the subject using techniques known in the art followed by the administration of the injectable composition to the blood vessel within or in close proximity to the mechanical device.

In one aspect, the injectable compositions and solids produced therefrom can be used to reinforce the inner wall of a blood vessel in the subject. The injectable composition can be introduced into the vessel at a sufficient volume to coat the inner lining of the vessel so that the vessel is not fully occluded. For example, the injectable composition can be injected into a blood vessel where there is an aneurysm. Here, the injectable composition can reduce or prevent the rupture of an aneurysm.

In one aspect, the injectable compositions and solids produced therefrom can be used to close or seal a puncture in a blood vessel in the subject. In one aspect, the injectable composition can be injected into a vessel at a sufficient amount to close or seal the puncture from within the vessel so that the vessel is not blocked. In another embodiment, the injectable composition can be applied to a puncture on the exterior surface of the vessel to seal the puncture.

In one aspect, the injectable compositions and solids and produced therefrom can be used to repair a number of different bone fractures and breaks. The solids and upon formation adhere to bone (and other minerals) through several mechanisms. The surface of the bone's hydroxyapatite mineral phase (Ca₅(PO₄)₃(OH)) is an array of both positive and negative charges. The negative groups present on the polyanion (e.g., phosphate groups) can interact directly with the positive surface charges or it can be bridged to the negative surface charges through the cationic groups on the polycation. Likewise, direct interaction of the polycation with the negative surface charges would contribute to adhesion. Alternatively, oxidized crosslinkers can couple to nucleophilic sidechains of bone matrix proteins.

Examples of such breaks include a complete fracture, an incomplete fracture, a linear fracture, a transverse fracture, an oblique fracture, a compression fracture, a spiral fracture, a comminuted fracture, a compacted fracture, or an open fracture. In one aspect, the fracture is an intra-articular fracture or a craniofacial bone fracture. Fractures such as intra-articular fractures are bony injuries that extend into and fragment the cartilage surface. The solids produced from the injectable compositions may aid in the maintenance of the reduction of such fractures, allow less invasive surgery, reduce operating room time, reduce costs, and provide a better outcome by reducing the risk of post-traumatic arthritis.

In other aspects, the injectable compositions and solids produced therefrom can be used to join small fragments of highly comminuted fractures. In this aspect, small pieces of fractured bone can be adhered to an existing bone. It is especially challenging to maintain reduction of the small fragments by drilling them with mechanical fixators. The smaller and greater the number of fragments the greater the problem. In one aspect, the injectable compositions may be injected in small volumes to create spot welds as described above in order to fix the fracture rather than filling the entire crack. The small biocompatible spot welds would minimize interference with healing of the surrounding tissue and would not necessarily have to be biodegradable. In this respect it would be similar to permanently implanted hardware.

In other aspects, the injectable compositions and solids produced therefrom can adhere a substrate to bone or other tissues such as, for example, cartilage, ligaments, tendons, soft tissues, organs, and synthetic derivatives of these materials. For example, implants made from titanium oxide, stainless steel, or other metals are commonly used to repair fractured bones. The injectable composition can be applied to the metal substrate, the bone, or both prior to adhering the substrate to the bone. Using the injectable composition and “spot welding” techniques described herein, the injectable compositions and solids produced therefrom can be used to position biological scaffolds in a subject. Small adhesive tacks composed of the injectable composition described herein would not interfere with migration of cells or transport of small molecules into or out of the scaffold. In certain aspects, the scaffold can contain one or more drugs that facilitate growth or repair of the bone and tissue. In other aspects, the scaffold can include drugs that prevent infection such as, for example, antibiotics. For example, the scaffold can be coated with the drug or, in the alternative, the drug can be incorporated within the scaffold so that the drug elutes from the scaffold over time.

It is also contemplated that the solids produced from the injectable compositions described herein can encapsulate, scaffold, seal, or hold one or more bioactive agents. Thus, the solid can be used as a delivery device or implantable drug depot.

The injectable composition and solids produced therefrom can be used in a variety of other surgical procedures. In one aspect, the injectable compositions and solids produced therefrom can be used to treat ocular wounds caused by trauma or by the surgical procedures. In one aspect, the injectable compositions and solids produced therefrom can be used to repair a corneal or schleral laceration in a subject. In other aspects, the injectable compositions can be used to facilitate healing of ocular tissue damaged from a surgical procedure (e.g., glaucoma surgery or a corneal transplant).

The methods disclosed in U.S. Published Application No. 2007/0196454, which are incorporated by reference, can be used to apply the injectable compositions described herein to different regions of the eye.

The injectable compositions and solids produced therefrom can be used to seal the junction between skin and an inserted medical device such as catheters, electrode leads, needles, cannulae, osseo-integrated prosthetics, and the like. Here, upon insertion and/or removal of the medical device is applied to the junction between the skin of the subject and the inserted medical device in order to seal the junction. Thus, the solid produced from the injectable composition prevent infection at the entry site when the device is inserted in the subject and subsequently forms a solid. In other aspects, the injectable compositions can be applied to the entry site of the skin after the device has been removed in order to expedite wound healing and prevent further infection.

In another aspect, the injectable compositions and solids produced therefrom can be used to prevent or reduce the proliferation of tumor cells during tumor biopsy. The method involves back-filling the track produced by the biopsy needle with the injectable compositions upon removal of the biopsy needle. In one aspect, the injectable compositions include an anti-proliferative agent that will prevent or reduce the potential proliferation of malignant tumor cells to other parts of the subject during the biopsy.

In another aspect, the injectable compositions and solids produced therefrom can be used to close or seal a puncture in an internal tissue or membrane. In certain medical applications, internal tissues or membranes are punctured, which subsequently have to be sealed in order to avoid additional complications. Alternatively, the injectable compositions and solids produced therefrom can be used to adhere a scaffold or patch to the tissue or membrane in order to seal the tissue, prevent further damage and facilitate wound healing.

In another aspect, the injectable compositions and solids produced therefrom can be used to seal a fistula in a subject. A fistula is an abnormal channel (pathway, tunnel) between an organ, vessel, or intestine and another structure such as, for example, skin. Fistulas are usually caused by injury or surgery, but they can also result from an infection or inflammation. Fistulas are generally a disease condition, but they may be surgically created for therapeutic reasons. In one aspect, the fistula is an enterocutaneous fistula (ECF). ECF is an abnormal channel that develops b-tween the intestinal tract or stomach and the skin. As a result, contents of the stomach or intestines leak through to the skin. Most ECFs occur after bowel surgery.

In other aspects, the injectable compositions and solids produced therefrom can prevent or reduce undesirable adhesion between two tissues in a subject, where the method involves contacting at least one surface of the tissue with the injectable composition.

In another aspect, the injectable composition and solids produced therefrom can anchor medical devices such as catheters in a blood vessel. The ability of the injectable compositions described herein to be converted to a solid or permits the anchoring of medical devices within the vessel. In one aspect, a catheter can be anchored to the inner wall of a blood vessel. In another aspect, two catheters can be inserted into a blood vessel and subsequently anchored to the inner wall of the vessel using the injectable composition. In this aspect, the catheter can be anchored in the vessel and be used as a delivery device for one or more bioactive agents for an extended period of time. The catheter can be removed from the embolus and the vessel. The resulting hole in the embolus can subsequently be filled with additional injectable composition described herein to enclose the hole and preserve the embolus.

The use of the injectable compositions to anchor delivery devices such as catheters within a blood vessel provides options and many potential benefits for the clinician. Targeted and focused delivery of bioactive agents and other materials to precise locations within the vasculature is a clinical challenge. Blood flow may carry agents downstream away from the intended target vessel and/or area resulting in a lower amount of bioactive agent or material, being injected into the target. In addition, any material that is released into a vessel and flows downstream away from the target may result in unintended consequences in the healthy, non-targeted, areas of the body.

The specific and controlled delivery of a bioactive agent or other materials can be delivered directly into the targeted area through the anchored catheter. Targeted infusion may increase the effectiveness of the bioactive agent where loss of bioactive agent due to flow in the vasculature system can be minimized. Furthermore, the catheter that is anchored in the vessel can act as a portal for the delivery of other materials and/or devices to a specific target vessel and/or area.

### Aspects

Aspect 1. An injectable composition comprising water, one or more polycationic polyelectrolytes and anionic counterions, one or more one polyanionic polyelectrolytes and cationic counterions, and a transient contrast agent, wherein the composition has an ion concentration that is (i) sufficient to prevent association of the polycationic polyelectrolytes and the polyanionic polyelectrolytes in water and (ii) greater than the concentration of ions in the subject, whereupon introduction of the composition into the subject a solid is produced in situ, and the transient contrast agent diffuses out of the solid.
Aspect 2. The composition of Aspect 1, wherein the transient contrast agent comprises an iodinated organic compound.
Aspect 3. The composition of Aspect 2, wherein the iodinated organic compound comprises iopamidol, iodixanol, iohexol, iopromide, iobtiridol, iomeprol, iopentol, iopamiron, ioxilan, iotrolan, iotrol and ioversol, iopanoate, diatrizoic acid, iothalamate, ioxaglate, or any combination thereof.
Aspect 4. The composition of Aspect 2, wherein the iodinated organic compound comprises an iodinated oil.
Aspect 5. The composition in any one of Aspects 1-4, wherein the concentration of the transient contrast agent in the injectable composition is from 10 mgI/mL to 1,000 mgI/mL.
Aspect 6. The composition in any one of Aspects 1-5, wherein up to 100% of the transient contrast agent diffuses out of the solid or gel from 5 minutes to 30 days.
Aspect 7. The composition in any one of Aspects 1-6, wherein the counterions comprise sodium and chloride ions.
Aspect 8. The composition in any one of Aspects 1-7, wherein the ion concentration in the injectable composition is 1.5 to 20 times greater than the ion concentration in the subject.
Aspect 9. The composition in any one of Aspects 1-8, wherein the polycationic polyelectrolyte is derived by dissolving a polycationic salt in water.
Aspect 10. The composition in any one of Aspects 1-8, wherein the polycationic polyelectrolyte is derived from a polycationic hydrochloride salt in water.
Aspect 11. The composition of Aspect 9 or 10, wherein the polycationic salt comprises a pharmaceutically-acceptable salt of a polyamine.
Aspect 12. The composition of Aspect 11, wherein the polyamine comprises two or more pendant amino groups, wherein the amino group comprises a primary amino group, a secondary amino group, tertiary amino group, a quaternary amine, an alkylamino group, a heteroaryl group, a guanidinyl group, an imidazolyl, or an aromatic group substituted with one or more amino groups.
Aspect 13. The composition of Aspect 11 or 12, wherein the pharmaceutically-acceptable salt of the polyamine comprises a dendrimer having 3 to 20 arms, wherein each arm comprises a terminal amino group.
Aspect 14. The composition Aspect 9 or 10, wherein the polycationic salt comprises a polyacrylate comprising two or more pendant amino groups, wherein the amino group comprises a primary amino group, a secondary amino group, tertiary amino group, a quaternary amine, an alkylamino group, a heteroaryl group, a guanidinyl group, an imidazolyl, or an aromatic group substituted with one or more amino groups.
Aspect 15. The composition of Aspect 9 or 10, wherein the polycationic salt comprises a pharmaceutically-acceptable salt of a biodegradable polyamine.
Aspect 16. The composition of Aspect 15, wherein the pharmaceutically-acceptable salt of the biodegradable polyamine comprises a polysaccharide, a protein, a peptide, a recombinant protein, a synthetic polyamine, a protamine, a branched polyamine, or an amine-modified natural polymer.
Aspect 17. The composition of Aspect 16, wherein the pharmaceutically-acceptable salt of the biodegradable polyamine comprises gelatin modified with an alkyldiamino compound.
Aspect 18. The composition of Aspect 9 or 10, wherein the polycationic salt comprises a pharmaceutically-acceptable salt of a protamine.
Aspect 19. The composition of Aspect 9 or 10, wherein the polycationic salt is a pharmaceutically-acceptable salt of salmine or clupein.
Aspect 20. The composition of Aspect 9 or 10, wherein the polycationic salt is a pharmaceutically-acceptable salt of natural polymer or a synthetic polymer containing two or more guanidinyl sidechains.
Aspect 21. The composition of Aspect 9 or 10, wherein the polycationic salt comprises a pharmaceutically-acceptable salt of a polyacrylate comprising two or more pendant guanidinyl groups.
Aspect 22. The composition of Aspect 9 or 10, wherein the polycationic salt comprises a pharmaceutically-acceptable salt of a homopolymer comprising pendant guanidinyl groups.
Aspect 23. The composition of Aspect 9 or 10, wherein the polycationic salt comprises a pharmaceutically-acceptable salt of a copolymer comprising two or more pendant guanidinyl groups.
Aspect 24. The composition of Aspect 9 or 10, wherein the polycationic salt comprises a pharmaceutically-acceptable salt of a synthetic polyguanidinyl copolymer comprising an acrylate, methacrylate, acrylamide, or methacrylamide backbone and two or more guanidinyl groups pendant to the backbone.
Aspect 25. The composition of Aspect 9 or 10, wherein the polycationic salt comprises a pharmaceutically-acceptable salt of a synthetic polyguanidinyl copolymer comprising the polymerization product between a monomer selected from the group consisting of an acrylate, a methacrylate, an acrylamide, a methacrylamide, or any combination thereof and a pharmaceutically-acceptable salt of compound of formula I

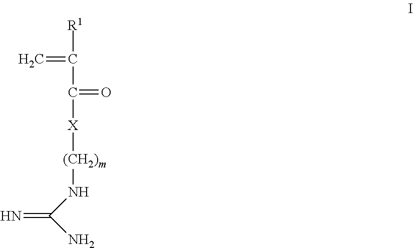

wherein R¹ is hydrogen or an alkyl group, X is oxygen or NR⁵, where R⁵ is hydrogen or an alkyl group, and m is from 1 to 10.

Aspect 26. The composition of Aspect 25, wherein the polycationic salt comprises a copolymerization product between the compound of formula I and an acrylate, a methacrylate, an acrylamide, or a methacrylamide,
Aspect 27. The composition of Aspect 25, wherein the polycationic salt comprises a copolymerization product between the compound of formula I and methacrylamide, N-(2-hydroxypropyl)methacrylamide (HPMA), N-[3-(N′-dicarboxymethyl)aminopropyl]methacrylamide (DAMA), N-(3-aminopropyl)methacrylamide, N-(1,3-dihydroxypropan-2-yl) methacrylamide, N-isopropylmethacrylamide, N-hydroxyethylacrylamide (HEMA), or any combination thereof.
Aspect 28. The composition of Aspect 25, wherein R¹ is methyl, X is NH, m is 3.
Aspect 29. The composition of Aspect 25, wherein the mole ratio of the guanidinyl monomer of formula Ito the comonomer is from 1:20 to 20:1.
Aspect 30. The composition of Aspect 25, wherein the polyguanidinyl copolymer has an average molar mass from 1 kDa to 1,000 kDa.
Aspect 31. The composition in any one of Aspects 1-30, wherein the polyanionic polyelectrolyte is derived by dissolving a polyanionic salt in water.
Aspect 32. The composition of Aspect 31, wherein the polyanionic salt comprises a pharmaceutically-acceptable salt of a synthetic polymer or a naturally-occurring polymer.
Aspect 33. The composition of Aspect 31 or 32, wherein the polyanionic salt comprises two or more carboxylate, sulfate, sulfonate, borate, boronate, phosphonate, or phosphate groups.
Aspect 34. The composition in any one of Aspects 31-33, wherein the polyanionic salt comprises a pharmaceutically-acceptable salt of a glycosaminoglycan or an acidic protein.
Aspect 35. The composition of Aspect 34, wherein the glycosaminoglycan comprises chondroitin sulfate, heparin, heparin sulfate, dermatan sulfate, keratin sulfate, or hyaluronic acid.
Aspect 36. The composition in any one of Aspects 31-35, wherein the polyanionic salt comprises a pharmaceutically-acceptable salt of a protein having a net negative charge at a pH of 6 or greater.
Aspect 37. The composition in any one of Aspects 31-33, wherein the polyanionic salt comprises a pharmaceutically-acceptable salt of a polymer comprising anionic groups pendant to the backbone of the polymer, incorporated in the backbone of the polymer backbone, or a combination thereof.
Aspect 38. The composition in any one of Aspects 31-33, wherein the polyanionic salt comprises a pharmaceutically-acceptable salt of a homopolymer or copolymer comprising two or more anionic groups.
Aspect 39. The composition in any one of Aspects 31-33, wherein the polyanionic salt is a copolymer comprising two or more fragments having the formula XI

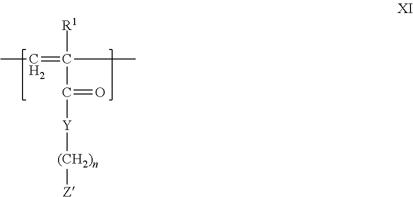

wherein R⁴ is hydrogen or an alkyl group;

n is from 1 to 10;

Y is oxygen, sulfur, or NR³⁰, wherein R³⁰ is hydrogen, an alkyl group, or an aryl group;

Z′ is a pharmaceutically-acceptable salt of an anionic group.

Aspect 40. The composition of Aspect 39, wherein Z′ is carboxylate, sulfate, sulfonate, borate, boronate, a substituted or unsubstituted phosphate or phosphonate.
Aspect 41. The composition of Aspect 40, wherein n is 2.
Aspect 42. The composition in any one of Aspects 31-33, wherein the polyanionic salt comprises a polyphosphate.
Aspect 43. The composition of Aspect 42, wherein the polyphosphate comprises a natural polymer or a synthetic polymer.
Aspect 44. The composition of Aspect 42, wherein the polyphosphate comprises polyphosphoserine.
Aspect 45. The composition of Aspect 42, wherein the polyphosphate comprises a polyacrylate comprising two or more pendant phosphate groups.
Aspect 46. The composition of Aspect 42, wherein the polyphosphate is the copolymerization product between a phosphate acrylate and/or phosphate methacrylate with one or more additional polymerizable monomers.
Aspect 47. The composition in any one of Aspects 31-33, wherein the polyanionic salt has from 10 to 1,000 phosphate groups.
Aspect 48. The composition in any one of Aspects 31-33, wherein the polyanionic salt comprises a pharmaceutically-acceptable salt of an inorganic polyphosphate, an organic polyphosphate, or a phosphorylated sugar.
Aspect 49. The composition of Aspect 48, wherein the polyanionic salt comprises a pharmaceutically-acceptable salt of inositol hexaphosphate.
Aspect 50. The composition of Aspect 48, wherein the polyanionic salt comprises a hexametaphosphate salt.
Aspect 51. The composition of Aspect 48, wherein the polyanionic salt comprises sodium hexametaphosphate.
Aspect 52. The composition in any one of Aspects 31-33, wherein the polyanionic salt comprises a pharmaceutically-acceptable salt of cyclic inorganic polyphosphate, a linear inorganic polyphosphate, or a combination thereof.
Aspect 53. The composition in any one of Aspects 31-33, wherein the polyanionic salt comprises a pharmaceutically-acceptable salt of a polyacrylate comprising two or more pendant phosphate groups.
Aspect 54. The composition in any one of Aspects 31-33, wherein the polyanionic salt comprises a pharmaceutically-acceptable salt of the copolymerization product between a phosphate or phosphonate acrylate or phosphate or phosphonate methacrylate with one or more additional polymerizable monomers.
Aspect 55. The composition in any one of Aspects 1-54, wherein the composition further comprises a reinforcing component, wherein the reinforcing component comprises natural or synthetic fibers, water-insoluble filler particles, a nanoparticle, or a microparticle.
Aspect 56. The composition of Aspect 55, wherein the reinforcing component comprises natural or synthetic fibers, water-insoluble filler particles, a nanoparticle, or a microparticle.
Aspect 57. The composition in any one of Aspects 1-56, wherein the composition further comprises one or more bioactive agents, wherein the bioactive agent comprises an antibiotic, a pain reliever, an immune modulator, a growth factor, an enzyme inhibitor, a hormone, a messenger molecule, a cell signaling molecule, a receptor agonist, an oncolytic virus, a chemotherapy agent, a receptor antagonist, a nucleic acid, a chemically-modified nucleic acid, or any combination thereof.
Aspect 58. The composition in any one of Aspects 1-57, wherein the composition has a viscosity of from 10 cp to 20,000 cp.
Aspect 59. The composition in any one of Aspects 1-58, wherein the total positive/negative charge ratio of the polycationic polyelectrolytes to the polyanionic polyelectrolytes is from 4 to 0.25 and the ion concentration in the composition is from 0.5 M to 2.0 M.
Aspect 60. The composition in any one of Aspects 1-59, wherein the concentration of the polycationic polyelectrolytes and the polyanionic polyelectrolytes is sufficient to yield a charge ratio of polycationic polyelectrolytes to polyanionic polyelectrolytes from 0.5:1 to 2:1.
Aspect 61. The composition in any one of Aspects 1-60, wherein the composition has a pH of 6 to 9.
Aspect 62. An injectable composition produced by the method comprising mixing at least one polycationic salt, at least one polyanionic salt, and a transient contrast agent in water, wherein the polycationic salt dissociates into polycationic polyelectrolytes and anionic counterions, and the polyanionic salt dissociates into polyanionic polyelectrolytes and cationic counterions, wherein the composition has an ion concentration that is (i) sufficient to prevent association of the polycationic polyelectrolytes and the polyanionic polyelectrolytes in water and (ii) greater than the concentration of ions in a subject, whereupon introduction of the composition into the subject a solid is produced in situ, and the transient contrast agent diffuses out of the solid.
Aspect 63. A method for producing a solid in a subject in situ comprising introducing into the subject the composition in any one of Aspects 1-62, wherein upon introduction of the composition into the subject the composition is converted to a solid in situ.
Aspect 64. A method for producing a bioactive eluting depot in the subject comprising injecting into the subject the composition in any one of Aspects 1-62.
Aspect 65. A method for reducing or inhibiting blood flow in a blood vessel of a subject comprising introducing into the vessel the composition in any one of Aspects 1-62, whereupon introduction of the composition into the vessel the composition is converted to a solid in situ within the vessel.
Aspect 66. The method of Aspect 65, wherein the method reduces or inhibits blood flow to a tumor, an aneurysm, a varicose vein, a vascular malformation, or a bleeding wound.
Aspect 67. The method of Aspect 65, wherein the method reinforces the inner wall of a blood vessel in the subject.
Aspect 68. A kit comprising

(a) a composition comprising a mixture of at least one polycationic salt and at least one polyanionic salt,

(b) a transient contrast agent, and

(c) instructions for making the injectable composition in any one of Aspects 1-62,

wherein the polycationic salt dissociates into polycationic polyelectrolytes and anionic counterions, and the polyanionic salt dissociates into polyanionic polyelectrolytes and cationic counterions, wherein the composition has an ion concentration that is (i) sufficient to prevent association of the polycationic polyelectrolytes and the polyanionic polyelectrolytes in water and (ii) greater than the concentration of ions in a subject, whereupon introduction of the composition into the subject a solid is produced in situ, and the transient contrast agent diffuses out of the solid.
Aspect 69. The kit of Aspect 68, wherein the composition comprising the mixture of the at least one polycationic salt and the at least one polyanionic salt is a dry powder.
Aspect 70. The kit of Aspect 68, wherein the composition comprising the mixture of the at least one polycationic salt and the at least one polyanionic salt further comprises water.
Aspect 71. The kit of Aspect 68, wherein the contrast agent is present in water.
Aspect 72. A kit comprising

(a) at least one polycationic salt,

(b) at least one polyanionic salt,

(c) a transient contrast agent, and

(d) instructions for making the injectable composition in any one of Aspects 1-62.

wherein the polycationic salt dissociates into polycationic polyelectrolytes and anionic counterions, and the polyanionic salt dissociates into polyanionic polyelectrolytes and cationic counterions, wherein the composition has an ion concentration that is (i) sufficient to prevent association of the polycationic polyelectrolytes and the polyanionic polyelectrolytes in water and (ii) greater than the concentration of ions in a subject, whereupon introduction of the composition into the subject a solid is produced in situ, and the transient contrast agent diffuses out of the solid.

### EXAMPLES

The following examples are put forth so as to provide those of ordinary skill in the art with a complete disclosure and description of how the compounds, compositions, and methods described herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. Numerous variations and combinations of reaction conditions, e.g. component concentrations, desired solvents, solvent mixtures, temperatures, pressures, and other reaction ranges and conditions can be used to optimize the product purity and yield obtained from the described process. Only reasonable and routine experimentation will be required to optimize such process conditions.

### Preparation of Poly N-(3-methacrylaminopropyl) Guanidinium Chloride (pGPMA-HCl )

The GPMA-HCl monomer was synthesized using procedures adapted from the literature[58, 59]. Briefly, a flask was charged with N-(3-aminopropyl) methacrylamide hydrochloride (APMA-HCl) and the inhibitor 4-methoxyphenol (1 wt.%, relative to APMA). DMF was added to dissolve APMA HCl at a concentration of 1 M. Triethylamine (TEA) (2.5 equivalents) was added to the flask and the mixture was stirred for 5 minutes under N₂ before/H-pyrazole-1-carboxamidine hydrochloride (1 equivalent) was added. The reaction proceeded at 20° C. under N₂. After 16 h, TEA.HC1 salts were separated from the reaction mixture by vacuum filtration. The GPMA monomer was extracted with diethyl ether 4 times and recovered as a dense oil. Finally, the monomer was dried under vacuum. The product was confirmed by proton and carbon NMR. 1H NMR (400 MHz, D2O): δ (ppm) 1.68 (q, CH₂—CH₂—CH₂), 1.77 (s, CH₃), 3.08 (m, CH₂—N), 3.18 (m, CH₂—N), 5.30 (s, ═CH₂), 5.55 (s, ═CH₂). ¹³C NMR: (400 MHz, D2O) δ (ppm) 17.74 (CH3), 27.62 (CH₂), 36.62 (CH₂—N), 38.71 (CH₂—N), 121.13 (C═CH₂), 138.83 (CH₂═C), 156.6 2(C), 171.55 (C═O). Formation of GPMA was also verified by ESI mass spectroscopy (185.1 Da).

A random copolymer of GPMA.HCl and methacrylamide (MA) was synthesized by free radical polymerization with a molar feed ratio of 60:40 (GPMA:MA). GPMA·HCl and MA monomers were dissolved in a 60:40 v:v water methanol mixture at a total monomer concentration of 1 M. 4,4′-Azobis(4-cyanovaleric acid was added as the initiator at 1-5% (w:v), depending on the desired molecular weight. The resulting mixture was septum sealed and degassed by bubbling for 1 hr with N₂. The reaction proceeded under N₂. The temperature was varied from 70-82° C. depending on the target M. The resulting solution was cooled, exposed to air, the polymer precipitated in acetone, then dissolved in water. The pH of the solution was adjusted to less than pH 6 using HCl. The polymer was purified by tangential flow filtration with deionized water. This process formed the hydrochloride salt at approximately a 1:1 stochiometric ratio of guanidinium to HCl. The polymer M_{w} was characterized by aqueous size exclusion chromatography (SEC) on an Aglient HPLC 1260 Infinity equipped with refractive index detector and a Wyatt miniDAWN TREOS light scattering detector. An elutent of 1 wt % acetic acid in 0.1 M LiBr (pH=3.3) was run at 1 mL/min on an Eprogen CATSEC 300 column. For M_{w} analysis using light scattering, the dn/dc value for p(GPMA-co-MA) was determined by injecting known stock solutions of PG ranging from 0.25-2 mg/mL at 1 mL/min into the Wyatt miniDAWN TREOS light scattering detector and measuring changes in intensity in response to concentration. The mole percent (mol %) GPMA was determined by relative integration of the CH₂-N groups (δ=2.8-3.2 ppm) on GPMA (4 total H) and the saturated hydrocarbon groups (δ=0.4-2.2 ppm) in the polymer backbone (5 total H's on both GPMA and MA) and polymer sidechain (2 H's on GPMA). P(GPMA-HCl) was also synthesized using an alternative method with equivalent results. First, a random copolymer of N-(3-aminopropyl) methacrylamide hydrochloride (APMA·HCl) and methacrylamide (MA) was synthesized by free radical polymerization at a fixed molar feed ratio of 60:40 (APMA:MA). The APMA-HCl and MA monomers were dissolved in a 60:40 v:v water methanol mixture at a total monomer concentration of 1 M. 4,4′-Azobis(4-cyanovaleric acid was added as the initiator at 1-5% (w: v), depending on the targeted polymer molecular weight. Reactions were done under N₂, with the reaction temperature varied from 70-82° C., depending on the target polymer M_{w}. The resulting solution was cooled, exposed to air, the p(APMA-co-MA-HCl) copolymer precipitated in acetone, then dissolved in water. Second, the sidechain primary amines of the p(APMA-co-MA)HCl copolymer were converted to guanidinium groups. The copolymer, p(APMA·HCl-co-MA), was dissolved in water at a concentration of ˜1 M. 1H-pyrazole-1-carboxamidine hydrochloride (1.15 equivalents relative to initial APMA) was added. Sodium carbonate was added to raise the pH of the reaction mixture to ˜9. The reaction proceeded for 14-28 hrs under N₂ at 25° C. Conversion of the APMA·HCl side chains to GPMA·HCl was >99% as determined using ¹H NMR. The product was then acidified to pH<6 with HCl, and tangential flow filtration with deionized water was used to purify the copolycation and associated counterions prior to lyophilization to produce the dry Cl− salt with approximately a 1:1 stoichiometric ratio of Cl⁻ ions to guanidinium sidechains.

### Preparation of Polyanionic Salts

Sodium Hexametaphosphate. Commercial sodium hexametaphosphate (NaₙMP) is a mixture of inorganic phosphate oligomers in sodium salt form, both cyclic and linear, usually containing 10-20 phosphorous atoms per chain [40-43]. In their fully ionized form, cyclic inorganic polyphosphates have the formula (PₙO₃ₙ)ⁿ⁻, while the linear form comprises (PₙO3ₙ₊₁)ⁿ⁺²⁻. Regardless of the whether the polyphosphate is linear or cyclic, each phosphorus atom has one weakly associated proton, with a pKa of ˜4.5 or less [40,44]. The end group protons of linear polyphosphates are dissociated between pH 4.5 and 9.5. Therefore, the charge density of NaₙMP at physiological pH (7.2-7.4) was calculated as one negative charge per phosphorous atom. Commercial NaₙMP was pH adjusted to 7.2-7.4 and dried by lyophilization to obtain the dry salt.

Poly(methacryloyloxyethyl phosphate) (pMOEP) sodium salts. Poly-MOEP was synthesized by free radical polymerization of MOEP (80 mol %), and methacrylic acid (20 mol %) in methanol (12.5 mg ml⁻¹ MOEP). The reaction was initiated with azobisisobutyronitrile (AIBN, 4.5 mol %) at 55° C., and proceeded for 15 h. The product was precipitated into acetone, then dissolved in water (200 ml H₂O per 10 g p-MOEP). The pH was adjusted to 7.4 with NaOH. The p-MOEP was purified by tangential flow filtration using a Millipore Pellicon 3 cassette filter with an Ultracel 10 kDa membrane.

The polymer was washed with 10 volumes of water during filtration. The product was lyophilized, and stored at ˜20 ° C. The resulting phosphate copolymer contained 83.5 mol % phosphate sidechains, 1.4 mol % HEMA, and 15.0 mol % MA sidechains, as determined by ¹H and ³¹P NMR. The molecular weight (M_{w}) and polydispersity index (PDI) of p-MOEP was determined by size exclusion chromatography (SEC) using an GPC Agilent system equipped with UV, RI and Wyatt MiniDawn Treos (light scattering) detectors. The AQ gel-OH mixed M (Agilent) column was equilibrated with 0.1 M sodium nitrate and 0.01M monosodium phosphate, pH 8.0. The average M_{w} and PDI were calculated using Wyatt MiniDawn ASTRA software to be 89 kDa and 1.6, respectively.

### Preparation of Injectable Compositions

Solutions of (poly)GPMA·HClₙ-co-MA (PG-HClₙ) and sodium hexametaphosphate (NaₙMP) were prepared by the addition of water to a mixture of dry PG·HClₙ and NaₙMP salts. Sequentially dissolving the polymers before mixing, as an alternative preparation method, resulted in final compositions with equivalent properties. Unless otherwise noted, solutions were prepared with 1:1 polymeric charge ratios, corresponding to a 2.65:1 PG-HClₙ to NaₙMP mass ratio. Solutions were prepared in which the PG-HClₙ concentrations were varied from 300-750 mg/mL using PG-HClₙ copolymers with average molecular weights (M_{w}) ranging from 19 to 53 kDa. The Cl⁻ and. Na⁺ concentrations of the solutions can be calculated from the concentrations (mol/L) and charge densities (mol/g) of the polymeric salts, PG-HClₙ and NaₙMP, respectively. The final polyelectrolyte concentrations and calculated concentrations of Na⁺ and Cl⁻ counterions in the polyelectrolyte solutions are shown in Table 1.

TABLE 1

| PG-HClₙ | NaₙMP | Calculated NaCl |
|---|---|---|
| Concentration | Concentration | Concentration |
| (mg/mL) | (mg/mL) | (mM) |
| 300 | 113 | 1080 |
| 350 | 132 | 1260 |
| 400 | 151 | 1440 |
| 450 | 170 | 1620 |
| 500 | 189 | 1800 |
| 550 | 207 | 1980 |
| 600 | 226 | 2160 |
| 650 | 245 | 2340 |
| 700 | 264 | 2520 |

The majority of the resulting injectable polyelectrolyte compositions were clear homogeneous solutions stable against macroscopic phase separation indefinitely. Some solutions using the lower M. (19 kDa) PG-HClₙ copolymer, at the lower end of the PG-HClₙ concentration range (350 mg/ml), turned cloudy and separated into two distinct liquid phases (complex coacervation). In these cases, stable homogeneous solutions were created by adding additional NaCl to increase the NaCl concentration to above the critical concentration for the particular polyelectrolyte solution. For example, the 19 kDa PG copolymer at 350 mg/ml phase separated into two liquid phases. With the addition of 180 mM NaCl to increase the total NaCl concentration to 1440 mM, equivalent to the NaCl concentration of a solution with 400 mg/ml PG, the solution became clear and stable against phase separation. All solutions solidified when injected into normal saline (150 mM NaCl).

### Preparation of Injectable Compositions with Transient Contrast Agents

Injectable compositions containing transient contrast agents were prepared using commercial solutions of non-ionic iodinated contrast media, diluted with water, to dissolve the dry polycationic and polyanionic salts. Solutions were prepared using non-ionic iohexol or iodixanol. The final concentration of the contrast agents ranged from 60 to 370 milligrams of iodine per milliliter (mgI/ml). The PG-HClₙ concentration was varied from 350-700 mg/mL with NaₙMP at a 1:1 charge ratio.

Injectable compositions with transient contrast agents were also prepared by emulsifying ethiodized oil (iodinated poppyseed oil) with the polyelectrolyte solutions using volume/volume ratios ranging from 2:1 to 1:2. The oil and polyelectrolyte solutions were loaded separated into syringes that were then connected with a female-female connector. The solutions were moved back and forth between syringes until thoroughly mixed immediately before delivery.

### Characterization of Injectable Compositions

### Liquid State Properties

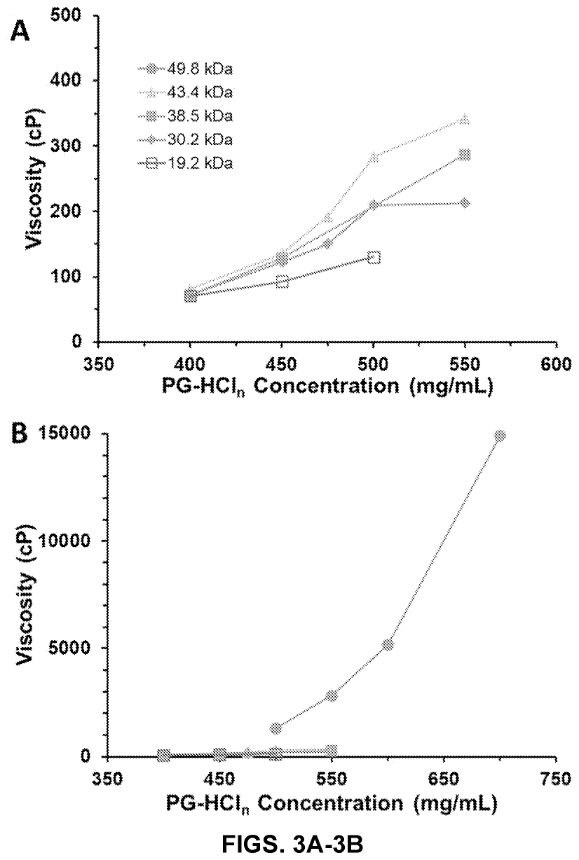

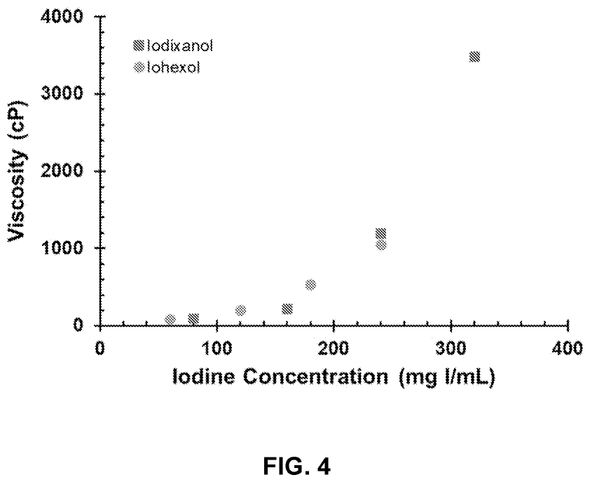

Viscosities of injectable compositions (ICs) were measured at 25° C. using a Brookfield Amrtek DV2T Viscometer with a small sample cup adaptor and CPA-41Z spindle. ICs were prepared with PG·HClₙ copolymers with M_{w} ranging from 19 to 50 kDa, and at PG·HClₙ concentrations of 350-700 mg/ml. All solutions were prepared with NaₙMP at a 1:1 polymeric charge ratio. The viscosity of the ICs ranged from 70 to 14,910 cP and increased with both PG·HClₙ molecular mass and concentration (FIG. 3). The viscosity of the ICs increased with both higher PG·HClₙ M_{w} and higher concentration. Increasing the concentration of PG·HCl from 350 mg/mL to 700 mg/mL, and M_{w} from 19 kDa to 50 kDa resulted in greater than 200-fold increase in viscosity (71 cP to 14910 cP). Thus, polymer concentration and molecular weight can be used to tune the viscosities for delivery through a wide array of microcatheters, needles, and cannulas. The range of viscosities can be extended using a wide range of M_{w}, polyelectrolyte concentrations, or mol % of ionic sidechains. The dependence of IC viscosity on non-ionic contrast agent concentration was similarly characterized. ICs were prepared with a fixed PG·HClₙ (M_{w} 42 kDa) concentration of 400 mg/mL and NaₙMP at a 1:1 polymeric charge ratio. As the concentrations of Iohexol and Iodixanol were separately varied from 60-240 and 80-320 mgI/ml, respectively, the IC viscosity increased from 60 cp to 3600 cp (FIG. 4).

The dependence of IC viscosity on PG·HClₙ concentration was evaluated using a fixed concentration of Iohexol (240 mgI/mL) and using PG·HClₙ (M_{w} 47 kDa) concentrations of 300 and 400 mg/mL. The total NaCl concentration was adjusted to 1440 mM in the 300 mg/ml solution (equal to the 400 mg/mL solution). Viscosities increased with increasing PG·HClₙ concentration, going from a viscosity of 451 cP at 300 mg/mL to a viscosity of 1010 cP at 400 mg/mL. Other non-ionic contrast agents and concentrations in similar trends in viscosity.

The viscosities of ICs emulsified with ethiodized oil at volume/volume ratios of 2:1 to 1:2 were all less than 100 cP. The viscosity of the 1:1 emulsion, for example, was 90 cp. The IC/oil emulsions were white and opaque, separating slowly over the course of minutes to hours. Upon delivery into saline, the emulsions formed a stiff, viscoelastic solid.

The results of viscosity characterization demonstrate that liquid state IC viscosity can be tuned using either or both the PG·HClₙ concentration and M_{w}, as well as the concentration of non-ionic contrast agent, to match the viscosity requirements of a specific application and delivery device.

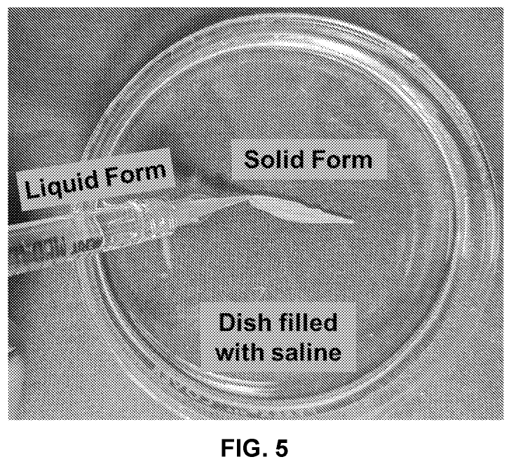

All of the ICs solidified when injected into 150 mM NaCl or physiological buffers, which was evaluated rheologically and visually. The solutions transition immediately from a clear solution into opaque viscoelastic solids. An example of an IC prepared with 80 mgI/mL of Iodixanol is shown in FIG. 5.

### Solid State Material Properties

The rheological properties of the solid state after injection of the ICs into unbuffered balanced salt solution (BSS), designed to mimic the ionic environment of blood, were characterized on a temperature-controlled rheometer (AR 2000ex, TA Instruments) at 37° C. Adhesive sandpaper was affixed to flat plate geometries (20 mm and 40 mm) to prevent slippage during measurements. ICs were prepared with two PG·HClₙ copolymers with M_{w} of 19 kDa and 53 kDa, and at two PG·HClₙ concentrations, 400 and 500 mg/ml, using NaₙMP at a 1:1 polymeric charge ratio. The ICs were injected on top of an inverted plate fixed in a circular mold. The mold containing the geometry and IC was submerged in BSS to solidify the IC. The system was allowed to equilibrate for 24 hrs before loading onto the rheometer. Oscillatory frequency sweeps from 0.1 to 1 Hz with a fixed strain of 1% was performed at 37° C. to examine viscoelastic properties.

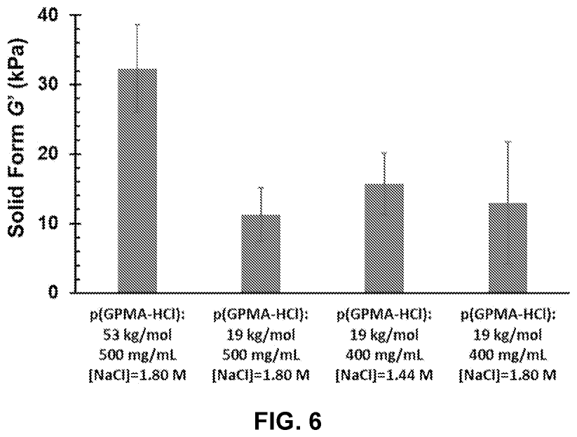

The elastic modulus (G′) at 1 Hz and 1% strain are shown in FIG. 6. The solidified ICs made with the 53 kDa PG·HClₙ copolymer had G′ values 2-4 fold higher than those made with the 19 kDa PG·HClₙ copolymer. The data demonstrate that higher PG·HClₙ copolymer M_{w} increases the stiffness (G′) of solidified ICs. The concentration of the 19 kDa PG·HClₙ copolymer or addition of NaCl to the liquid form had little effect on the final stiffness of the solid form.

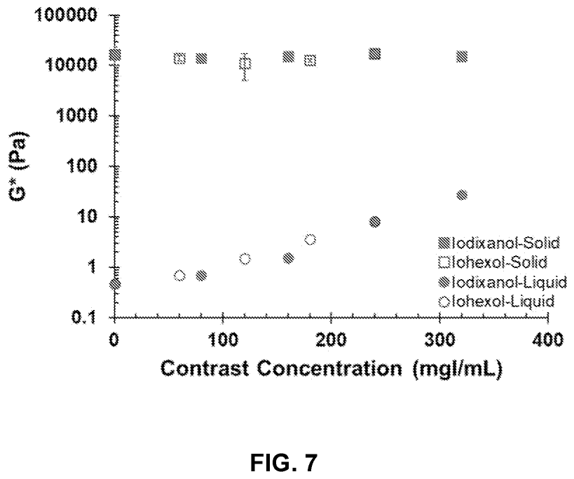

The effect of non-ionic contrast agents on the rheological properties of the ICs were similarly characterized. The complex modulus (G*) at 1 hz and 1% strain for a range of Iohexol and Iodixanol concentrations, for both the liquid and solidified forms, are shown in FIG. 7. Increasing concentrations of both contrast agents increased G* from 0.5 up to 27 Pa. The G* of the solid forms were around 20,000 for both contrast agents and at all concentrations. This is a 3-4 order of magnitude increase compared to the liquid forms. One-way ANOVA revealed no statistically significant differences between the various solid forms (p>0. 05).

The results demonstrate that the rheological properties of the solidified form of the injectable polyelectrolyte solutions are more than adequate to produce effective occlusions of blood vessels. For comparison, natural fibrin clots have moduli of around 600 Pa, which is sufficient to create stable occlusion of blood vessels. Likewise, moduli of the solidified ICs are at least an order of magnitude higher than several other systems that have demonstrated efficacy in animal models [47, 60, 61].

### Duration of Non-Ionic Contrast Agents in the Solid State

The time course of non-ionic contrast agents diffusing out of solidified ICs were evaluated by micro-CT in gelatin tissue phantoms. Gelatin powder (Porcine skin Type A, 300 g bloom, 5 wt/v%) was heated in water to 45° C. Cylindrical tissue phantoms were created by adding the warm gelatin solution to a mold comprising a 2.5 cm diameter outside tube and a central interior 2 mm diameter tube. The tubes were lightly coated with olive oil to facilitate removal of the phantom. One end was sealed with paraffin and the warm gelatin solution was added to the outside tube. After cooling to room temperature, the central tube was removed leaving an empty 2 mm central tunnel in the solid gelatin cylinder.

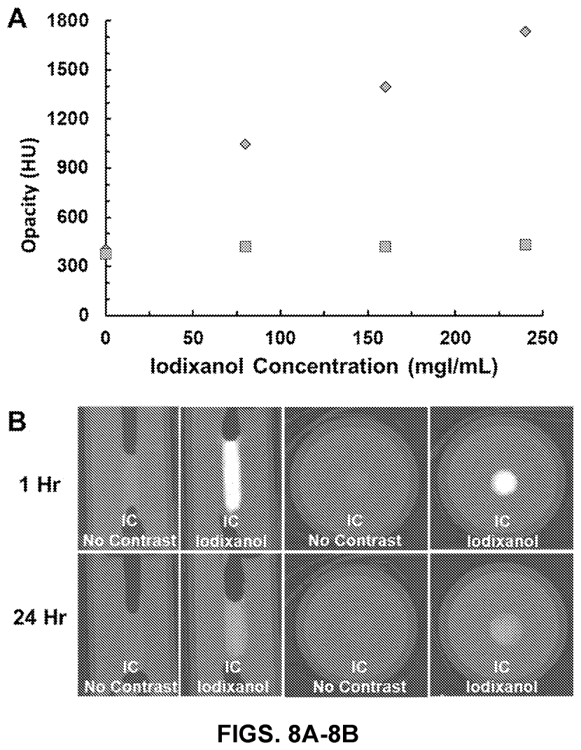

Contrast-containing ICs were prepared by dissolving dry PG-HClₙ (40 kDa, 400 mg/ml) and 1:1 NaₙMP in Iohexol or Iodixanol solutions diluted with water to concentrations ranging from 0 to 270 mgI/mL. The ICs (50 μL) were injected into the 2 mm diameter tunnel of molded gelatin cylinders. After IC solidification, the gelatin phantoms were removed from the mold and wrapped in polyethylene film. The phantoms were imaged by micro-CT within 1 hr of preparation. Radiopacity (HU) of the solidified IC within the phantom as a function of iodixanol concentration at 1 hr and 24 hr are shown in FIG. 8A. Initial radiopacity increased from 376 HU in IC gelatin phantoms with 0 iodixanol to 1734 HU at 270 MgI/ml iodixanol. For comparison, the mid-range radiopacity of cortical bone is approximately 1100 HU.

The phantoms were re-imaged after 24 hr. For all three concentrations of iodixanol, the radiopacity had decreased to nearly the level of the sample without iodixanol (423-433 HU). Vertical and axial images of the phantoms containing 0 and 68 mgI/ml, at 1 and 24 hr, are shown in FIG. 8B. At 1 hr, the solidified IC-Iodixanol plug is radiopaque and easily distinguishable from the gelatin phantom. The solidified IC plug with 0 iodixanol has low radiopacity, barely higher than the surrounding gelatin phantom. After 24 hours, the radiopacity of the solidified IC-Iodixanol plug has markedly decreased to be only slightly more radiopacity than the surrounding gelatin phantom. Similar results were obtained with both iohexol and ethiodized oil. The results demonstrate that non-ionic contrast agents are still highly visible after 1 hr, but have largely diffused out of the solidified IC into the surrounding tissue phantom within 24 hr. Similar time courses are expected in blood vessels and living tissues.

### Animal Studies

### Swine In Vivo Embolization Model

The duration of fluoroscopic visibility and embolization efficacy of several ICs was examined in swine models, which are widely used to test novel embolic agents. Arterial sites involved access through the femoral artery using the Seldinger technique. From there, the catheter was guided using standard techniques into sites originating from the renal and hepatic arteries. The injectable composition was delivered through the catheter. Angiograms were captured before and after embolization using either the same catheter or a base catheter. The site was then assessed as Fully Occluded, Partially Occluded, or Not Occluded. Follow-up imaging of the delivery site was conducted at 1 day and 7 days post embolization. Angiography was also performed immediately after embolization and at 7 days post embolization when vessel access could be obtained.

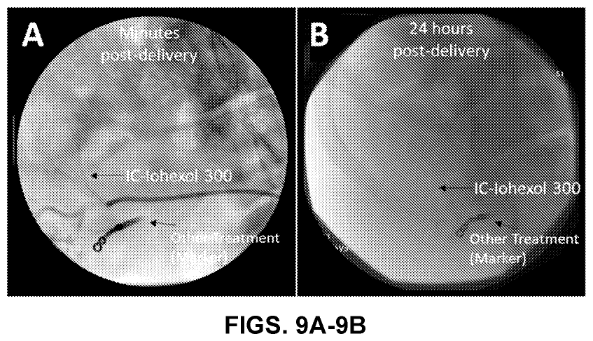

An IC was prepared by dissolving PG-HClₙ (300 mg/mL) and NaₙMP at a 1:1 polymeric charge ratio in a 300 mgI/mL solution of Iohexol. Access to a subbranch of the renal artery was obtained with a 4F catheter. The IC was readily visible under fluoroscopy (FIG. 9) distally penetrating into the renal vasculature. After delivery of 0.3 mL of the IC, the occlusion was confirmed by angiography. The target region remained completely occluded. At 24 hours after embolization, follow-up fluoroscopic imaging revealed that radiopacity had dissipated out of the embolization site, confirming findings from the bench top gelatin phantom experiments. Angiography performed 7 days post-embolization showed that the target region remained occluded. Similar results were obtained with samples prepared in various concentrations of Iohexol (180, 240, 300 mgI/mL) and Iodixanol (270 mgI/mL).

### Iodinated Oil-based Contrast

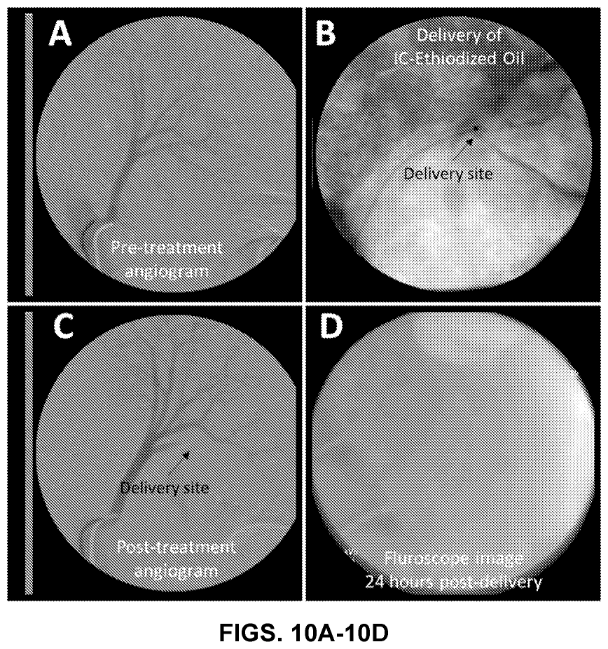

An IC prepared by dissolving PG-HClₙ (400 mg/mL) and NaₙMP at a 1:1 charge ratio was mixed with Lipiodol at a 1:1 ratio just prior to delivery. Access to the caudal pole of the kidney was obtained with a 2.8 F microcatheter. The mixture produced a stable, opaque emulsion, which was delivered through the catheter into the target pole of the kidney (FIG. 10). Approximately 0.3 mL of the embolic IC was delivered, which was readily visible under fluoroscopy. An angiogram immediately post deployment showed full occlusion, which remained occluded prior to termination after 7 days. Additional follow-up fluoroscopic imaging showed no discernable radiopacity at 24 hours and 7 days. These results showed that PE embolic agents could be formulated into emulsions with oily contrast agents and maintain the ability to occlude the vessel. The results also confirmed the transient radiopacity with oil-based contrast agents.

### Summary

The injectable compositions formed in combination with non-ionic contrast or iodinated oils provided temporary radiopacity, of intermediate duration between rapidly dissipating agents and permanent agents. Contrast persisted for hours in both benchtop and animal models. This intermediate duration radiopacity provides utility in that it does not interfere in any subsequent imaging, including CT or future treatment of nearby targets. It also allows electrocautery to be performed on the embolized tissue, in contrast to embolization agents with metallic contrast. In contrast to other embolic agents with transient radiopacity that diminishes in seconds to a few minutes, the iodinated organic contrast in the injectable compositions persists for a period of hours. By allowing the delivered embolic to remain visible throughout the duration of the procedure, this property eliminates many of the disadvantages of immediately dissipating contrast, resulting in better confirmation of embolic placement and providing guidance for subsequent injections if necessary. Furthermore, the elimination of dark-colored metallic particles prevents visible skin tattooing in superficial applications.

The ICs can be produced with a variety of contrast agents. The ICs can be formed by direct dissolution of the polycationic and polyanionic salts in aqueous solutions of non-ionic contrast media. The addition of non-ionic contrast to the ICs increased viscosity with increasing contrast concentration, providing an additional parameter for tuning viscosity. Mixing of aqueous solutions of polycationic and polyanionic salts with iodinated oils produced ICs as oil-in-water emulsions that had low viscosities and solidified when delivered into solutions near physiological ionic strength. These solutions and emulsions had viscosities appropriate for transcatheter embolization and demonstrated acceptable performance in animal models.

The viscosity of the ICs can be tuned by modifying the M_{w} and concentration of the polyelectrolytes. The viscosity of the injectable compositions can span more than three orders of magnitude (10¹-10⁴ cP). The low viscosity solutions are deliverable through narrow (0.013″ ID) and long (150 cm) microcatheters. Higher viscosity formulations (up to 15,000 cP) may provide greater feedback, control, and effective embolization through larger microcatheters, cannulas, or needles.

Throughout this application, various publications are referenced. The disclosures of these publications in their entireties are hereby incorporated by reference into this application in order to more fully describe the compounds, compositions, and methods described herein.

Various modifications and variations can be made to the compounds, compositions, and methods described herein. Other aspects of the compounds, compositions, and methods described herein will be apparent from consideration of the specification and practice of the compounds, compositions, and methods disclosed herein. It is intended that the specification and examples be considered as exemplary.

### REFERENCES

[1] F. Y. Yap, B. O. Omene, M. N. Patel, T. Yohannan, J. Minocha, M. G. Knuttinen, C. A. Owens, J. T. Bui, R. C. Gaba, Transcatheter embolotherapy for gastrointestinal bleeding: a single center review of safety, efficacy, and clinical outcomes, Dig. Dis. Sci. 58(7) (2013) 1976-1984.
[2] R. S. Ramaswamy, H. W. Choi, H. C. Mouser, K. H. Narsinh, K. C. McCammack, T. Treesit, T. B. Kinney, Role of interventional radiology in the management of acute gastrointestinal bleeding, World J. Radiol. 6(4) (2014) 82-92.
[3] L. Defreyne, P. Vanlangenhove, M. De Vos, P. Pattyn, G. Van Maele, J. Decruyenaere, R. Troisi, M. Kunnen, Embolization as a first approach with endoscopically unmanageable acute nonvariceal gastrointestinal hemorrhage, Radiology 218(3) (2001) 739-48.
[4] J. Rabkin, V. Astafjev, L. Gothman, Y. Grigorjev, Transcatheter embolization in the management of pulmonary hemorrhage, Radiology 163(2) (1987) 361-365.
[5] M. K. Kolber, P. A. Shukla, A. Kumar, J. E. Silberzweig, Ethylene vinyl alcohol copolymer (onyx) embolization for acute hemorrhage: a systematic review of peripheral applications, J. Vasc. Interv. Radiol. 26(6) (2015) 809-15.

### [6] M. Waqas, K. Vakhari, P. V. Weimer, E. Hashmi, J. M. Davies, A. H. Siddiqui, Safety and Effectiveness of Embolization for Chronic Subdural Hematoma: Systematic Review and Case Series, World Neurosurg. 126 (2019) 228-236.

### [7] T. W. Link, S. Boddu, S. M. Paine, H. Kamel, J. Knopman, Middle Meningeal Artery Embolization for Chronic Subdural Hematoma: A Series of 60 Cases, Neurosurgery 85(6) (2019) 801-807.

[8] W. H.-C. Jeske, T. R. Larndorfer, T. D. Krappinger, T. R. Anal, T. M. Klingensmith, T. C. Lottersberger, T. M. Dtinser, T. M. Blauth, T. S. Falle, T. C. Dallapozza, Management of Hemorrhage in Severe Pelvic Injuries, The Journal of Trauma: Injury, Infection, and Critical Care 68(2) (2010) 415-420.
[9] W. J. van Rooij, M. Sluzewski, G. N. Beute, Brain AVM Embolization with Onyx, Am. J. Neuroradiol. 28(1) (2007) 172-177.
[10] J. Van Beijnum, H. B. Van Der Worp, D. R. Buis, R. A.-S. Salman, L. J. Kappelle, G. J. Rinkell, J. W. B. Van Der Sprenkel, W. P. Vandertop, A. Algra, C. J. Klijn, Treatment of Brain Arteriovenous Malformations: A Systematic Review and Meta-Analysis, J. Am. Med. Assoc. 306(18) (2012) 2011-2019.
[11] Y. C. Hu, C. B. Newman, S. R. Dashti, F. C. Albuquerque, C. G. McDougall, Cranial dural arteriovenous fistula: transarterial Onyx embolization experience and technical nuances, J. Neurointerv. Surg. 3(1) (2011) 5-13.
[12] I. Linfante, A. K. Wakhloo, Brain aneurysms and arteriovenous malformations advancements and emerging treatments in endovascular embolization, Stroke 38(4) (2007) 1411-1417.
[13] J. Urbano, M. Cabrera, A. Alonso-Burgos, Sclerosis and varicocele embolization with N-butyl cyanoacrylate: experience in 41 patients, Acta Radiol. 55(2) (2014) 179-185.
[14] A. D. Talenfeld, A. K. Sista, D. C. Madoff, Transarterial therapies for primary liver tumors, Surg. Oncol. Clin. N. Am. 23(2) (2014) 323-351.
[15] L. Bester, B. Meteling, D. Boshell, T. C. Chua, D. L. Morris, Transarterial chemoembolisation and radioembolisation for the treatment of primary liver cancer and secondary liver cancer: a review of the literature, J. Med. Imaging Radiat. Oncol. 58(3) (2014) 341-352.
[16] M. A. Lazzaro, A. Badruddin, O. O. Zaidat, Z. Darkhabani, D. J. Pandya, J. R. Lynch, Endovascular embolization of head and neck tumors, Front. Neurol. 2 (2011).
[17] D.T. Ginat W. E. Saad, U. C. Turba, Transcatheter renal artery embolization: clinical applications and techniques, Tech Vasc Intery Radiol 12(4) (2009) 224-39.
[18] P.H. Lin, T. T. Terramani, R. L. Bush, T. E. Keane, R. G. Moore, A. B. Lumsden, Concomitant intraoperative renal artery embolization and resection of complex renal carcinoma, J. Vasc. Surg. 38(3) (2003) 446-450.
[19] B. J. May, A. D. Talenfeld, D. C. Madoff, Update on Portal Vein Embolization: Evidence-based Outcomes, Controversies, and Novel Strategies, J. Vasc. Interv. Radiol. 24(2) (2013) 241-254.
[20] A. Abulkhir, P. Limongelli, A. J. Healey, O. Damrah, P. Tait, J. Jackson, N. Habib, L. R. Jiao, Preoperative Portal Vein Embolization for Major Liver Resection: A Meta-Analysis, Ann. Surg. 247(1) (2008) 49-57.

### [21] P. Jones, B. P. Rai, R. Nair, B. K. Somani, Current Status of Prostate Artery Embolization for Lower Urinary Tract Symptoms: Review of World Literature, Urology 86(4) (2015) 676-81.

### [22] N. Hafezi-Nejad, C. R. Bailey, C. R. Weiss, Bariatric Embolization: A Narrative Review of Clinical Data From Human Trials, Tech Vasc Intery Radiol 23(1) (2020) 100658.

[23] Y. Okuno, A. M. Korchi, T. Shinjo, S. Kato, Transcatheter arterial embolization as a treatment for medial knee pain in patients with mild to moderate osteoarthritis, Cardiovasc. Intervent. Radiol. 38(2) (2015) 336-43.
[24] M. Lubarsky, C. E. Ray, B. Funaki, Embolization agents—Which one should be used when? Part 1: Large-vessel embolization, Semin. Intervent. Radiol. 26(4) (2009) 352-357.
[25] M. Lubarsky, C. Ray, B. Funaki, Embolization agents—Which one should be used when? Part 2: Small-vessel embolization, Semin. Intervent. Radiol. 27(1) (2010) 99-104.
[26] W. Taki, Y. Yonekawa, H. Iwata, A. Uno, K. Yamashita, H. Amemiya, A new Liquid Material for Embolization of Arteriovenous Malformations, Am. J. Neuroradiol. 11(1) (1990) 163-168.
[27] J. S. Pollak, R. I. White, The use of cyanoacrylate adhesives in peripheral embolization, J. Vasc. Interv. Radiol. 12(8) (2001) 907-913.
[28] G. M. Debrun, V. A. Aletich, H. Shownkeen, J. Ausman, Glued Catheters during Embolisation of Brain AVMs with Acrylic Glue, Interv. Neuroradiol. 3(1) (1997) 13-9.
[29] S. Paramasivam, D. Altschul, S. Ortega-Gutiarrez, J. Fifi, A. Berenstein, N-butyl cyanoacrylate embolization using a detachable tip microcatheter: initial experience, J. Neurointerv. Surg. 7(6) (2015) 458-61.
[30] A. S. Puri, R. Rahbar, J. Dearden, R. J. Graham, C. Lillehei, D. B. Orbach, Stretched and Sheared Microcatheter Retained after Onyx Embolization of Infantile Myofibromatosis, Interv. Neuroradiol. 17(2) (2011) 261-266.
[31] A. I. Qureshi, N. Mian, H. Siddiqi, M. H. Qureshi, A. M. Malik, M. Rauf Afzal, A. A. Khan, M. F. K. Suri, Occurrence and Management Strategies for Catheter Entrapment with Onyx Liquid Embolization, J. Vasc. Interv. Neurol. 8(3) (2015) 37-41.
[32] S. Vaidya, K. R. Tozer, J. Chen, An Overview of Embolic Agents, Semin. Intervent. Radiol. 25(3) (2008) 204-215.
[33] R. J. Rosen, S. Contractor, The use of cyanoacrylate adhesives in the management of congenital vascular malformations, Semin. Intervent. Radiol. 21(1) (2004) 59-66.
[34] F. Mottu, A. Laurent, D. A. Rüfenacht, E. Doelker, Organic solvents for pharmaceutical parenterals and embolic liquids: a review of toxicity data, PDA J. Pharm. Sci. Technol. 54(6) (2000) 456-469.
[35] M. Guimaraes, M. Wooster, Onyx (Ethylene-vinyl Alcohol Copolymer) in Peripheral Applications, Semin. Intervent. Radiol. 28(3) (2011) 350-356.
[36] F. Numan, A. Ömeroglŭ, B. Kara, M. Cantasdemir, İ. Adaletli, F. Kantarci, Embolization of Peripheral Vascular Malformations with Ethylene Vinyl Alcohol Copolymer (Onyx), J. Vasc. Interv. Radiol. 15(9) (2004) 939-946.
[37] S. L. Blackburn, Y. Kadkhodayan, W. Z. Ray, G. J. Zipfel, D. T. Cross, 3rd, C. J. Moran, C. P. Derdeyn, Onyx is associated with poor venous penetration in the treatment of spinal dural arteriovenous fistulas, J. Neurointerv. Surg. 6(7) (2014) 536-40.
[38] J. Raymond, A. Metcalfe, I. Salazkin, A. Schwarz, Temporary vascular occlusion with poloxamer 407, Biomaterials 25(18) (2004) 3983-3989.
[39] H. H. Bearat, M. C. Preul, B. L. Vernon, Cytotoxicity, In Vitro models and Preliminary In Vivo study of dual physical and chemical gels for endovascular embolization of cerebral aneurysms, J. Biomed. Mater. Res. 101(9) (2013) 2515-25.
[40] A. Poursaid, R. Price, A. Tiede, E. Olson, E. Huo, L. McGill, H. Ghandehari, J. Cappello, In situ gelling silk-elastinlike protein polymer for transarterial chemoembolization, Biomaterials 57 (2015) 142-52.
[41] M. M. Jensen, Z. B. Barber, N. Khurana, K. J. Isaacson, D. Steinhauff, B. Green, J. Cappello, A. Pulsipher, H. Ghandehari, J. A. Alt, A dual-functional Embolization-Visualization System for Fluorescence image-guided Tumor Resection, Theranostics (2020) 4530-4543.
[42] A. Poursaid, M. M. Jensen, I. Nourbakhsh, M. Weisenberger, J. W. Hellgeth, S. Sampath, J. Cappello, H. Ghandehari, Silk-Elastinlike Protein Polymer Liquid Chemoembolic for Localized Release of Doxorubicin and Sorafenib, Mol. Pharm. 13(8) (2016) 2736-2748.
[43] A. S. Sawhney, H. Claesson, R. Lareau, D. Billings, Embolic compositions and methods, Google Patents, 2019.
[44] T. A. Becker, D. R. Kipke, T. Brandon, Calcium alginate gel: a biocompatible and mechanically stable polymer for endovascular embolization, J. Biomed. Mater. Res. 54(1) (2001) 76-86.
[45] A. Momeni, E. M. Valliant, E. P. Brennan-Pierce, J. J. S. Shankar, R. Abraham, P. Colp, M. J. Filiaggi, Developing an In Situ Forming Polyphosphate Coacervate as a New Liquid Embolic Agent: From Experimental Design to Pilot Animal Study, Acta Biomater. 32 (2016) 286-297.

### [46] L. Weng, N. Rostambeigi, N. D. Zantek, P. Rostamzadeh, M. Bravo, J. Carey, J. Golzarian, An In Situ Forming Biodegradable Hydrogel-based Embolic Agent for Interventional Therapies, Acta Biomater. 9(9) (2013) 8182-8191.

[47] R. K. Avery, H. Albadawi, M. Akbari, Y. S. Zhang, M. J. Duggan, D. V. Sahani, B. D. Olsen, A. Khademhosseini, R. Oklu, An injectable shear-thinning biomaterial for endovascular embolization, Sci. Transl. Med. 8(365) (2016) 156.
[48] J. Hu, H. Albadawi, B. W. Chong, A. R. Deipolyi, R. A. Sheth, A. Khademhosseini, R. Oklu, Advances in Biomaterials and Technologies for Vascular Embolization, Adv. Mater. 31(33) (2019) 1901071.
[49] K. S. Sakariassen, L. Orning, V. T. Turitto, The impact of blood shear rate on arterial thrombus formation, Future Sci OA 1(4) (2015) FSO30-FSO30.
[50] Q. Wang, J. B. Schlenoff, The polyelectrolyte complex/coacervate continuum, Macromolecules 47(9) (2014) 3108-3116.
[51] R. Chollakup, W. Smitthipong, C. D. Eisenbach, M. Tirrell, Phase behavior and coacervation of aqueous poly (acrylic acid)—poly (allylamine) solutions, Macromolecules 43(5) (2010) 2518-2528.
[52] R. Chollakup, J. B. Beck, K. Dirnberger, M. Tirrell, C. D. Eisenbach, Polyelectrolyte Molecular Weight and Salt Effects on the Phase Behavior and Coacervation of Aqueous Solutions of Poly(acrylic acid) Sodium Salt and Poly(allylamine) Hydrochloride, Macromolecules 46(6) (2013) 2376-2390.
[53] H. Shao, K. N. Bachus, R. J. Stewart, A Water-Borne Adhesive Modeled after the Sandcastle Glue of P. californica, Macromol. Biosci. 9(5) (2009) 464-471.
[54] H. Shao, R. J. Stewart, Biomimetic underwater adhesives with environmentally triggered setting mechanisms, Adv. Mater. 22(6) (2010) 729-733.

### [55] R. A. Ghostine, R. F. Shamoun, J. B. Schlenoff, Doping and Diffusion in an Extruded Saloplastic Polyelectrolyte Complex, Macromolecules 46(10) (2013) 4089-4094.

[56] H. H. Hariri, A. M. Lehaf, J. B. Schlenoff, Mechanical properties of osmotically stressed polyelectrolyte complexes and multilayers: Water as a plasticizer, Macromolecules 45(23) (2012) 9364-9372.
[57] C. E. Sing, Development of the Modern Theory of Polymeric Complex Coacervation, Adv. Colloid Interface Sci. 239 (2017) 2-16.
[58] Y. Yonamine, K. Yoshimatsu, S.-H. Lee, Y. Hoshino, Y. Okahata, K. J. Shea, Polymer Nanoparticle—Protein Interface. Evaluation of the Contribution of Positively Charged Functional Groups to Protein Affinity, ACS Appl. Mater. Interfaces 5(2) (2013) 374-379.
[59] M. S. Bernatowicz, Y. Wu, G. R. Matsueda, 1H-pyrazole-1-carboxamidine hydrochloride an attractive reagent for guanylation of amines and its application to peptide synthesis, J. Org. Chem. 57(8) (1992) 2497-2502.
[60] F. Zhou, L. Chen, Q. An, L. Chen, Y. Wen, F. Fang, W. Zhu, T. Yi, Novel Hydrogel Material as a Potential Embolic Agent in Embolization Treatments, Sci. Rep. 6 (2016) 32145.
[61] X. Li, W. Liu, G. Ye, B. Zhang, D. Zhu, K. Yao, Z. Liu, X. Sheng, Thermosensitive N-isopropylacrylamide-N-propylacrylamide-vinyl pyrrolidone terpolymers: Synthesis, characterization and preliminary application as embolic agents, Biomaterials 26(34) (2005) 7002-7011.
[62] J. W. Weisel, The mechanical properties of fibrin for basic scientists and clinicians, Biophys. Chem. 112(2) (2004) 267-276.
[63] P. Riha, X. Wang, R. Liao, J. F. Stoltz, Elasticity and fracture strain of whole blood clots, Clin. Hemorheol. Microcirc. 21(1) (1999) 45-49.
[64] C. Storm, J. J. Pastore, F. C. MacKintosh, T. C. Lubensky, P. A. Janmey, Nonlinear elasticity in biological gels, Nature 435(7039) (2005) 191-194.

What is claimed:

1. An injectable composition comprising water, one or more polycationic polyelectrolytes and anionic counterions, one or more one polyanionic polyelectrolytes and cationic counterions, and a transient contrast agent, wherein the composition has an ion concentration that is (i) sufficient to prevent association of the polycationic polyelectrolytes and the polyanionic polyelectrolytes in water and (ii) greater than the concentration of ions in the subject, whereupon introduction of the composition into the subject a solid is produced in situ, and the transient contrast agent diffuses out of the solid.

2. The composition of claim 1, wherein the transient contrast agent comprises an iodinated organic compound.

3. The composition of claim 2, wherein the iodinated organic compound comprises iopamidol, iodixanol, iohexol, iopromide, iobtiridol, iomeprol, iopentol, iopamiron, ioxilan, iotrolan, iotrol and ioversol, iopanoate, diatrizoic acid, iothalamate, ioxaglate, or any combination thereof.

4. The composition of claim 2, wherein the iodinated organic compound comprises an iodinated oil.

5. The composition of claim 1, wherein the concentration of the transient contrast agent in the injectable composition is from 10 mgI/mL to 1,000 mgI/mL.

6. The composition of claim 1, wherein up to 100% of the transient contrast agent diffuses out of the solid or gel from 5 minutes to 30 days.

7. The composition of claim 1, wherein the counterions comprise sodium and chloride ions.

8. The composition of claim 1, wherein the ion concentration in the injectable composition is 1.5 to 20 times greater than the ion concentration in the subject.

9. The composition of claim 1, wherein the polycationic polyelectrolyte is derived by dissolving a polycationic salt in water.

10. The composition of claim 1, wherein the polycationic polyelectrolyte is derived from a polycationic hydrochloride salt in water.

11. The composition of claim 9, wherein the polycationic salt comprises a pharmaceutically-acceptable salt of a polyamine.

12. The composition of claim 11, wherein the polyamine comprises two or more pendant amino groups, wherein the amino group comprises a primary amino group, a secondary amino group, tertiary amino group, a quaternary amine, an alkylamino group, a heteroaryl group, a guanidinyl group, an imidazolyl, or an aromatic group substituted with one or more amino groups.

13. The composition of claim 11, wherein the pharmaceutically-acceptable salt of the polyamine comprises a dendrimer having 3 to 20 arms, wherein each arm comprises a terminal amino group.

14. The composition claim 9, wherein the polycationic salt comprises a polyacrylate comprising two or more pendant amino groups, wherein the amino group comprises a primary amino group, a secondary amino group, tertiary amino group, a quaternary amine, an alkylamino group, a heteroaryl group, a guanidinyl group, an imidazolyl, or an aromatic group substituted with one or more amino groups.

15. The composition of claim 9, wherein the polycationic salt comprises a pharmaceutically-acceptable salt of a biodegradable polyamine.

16. The composition of claim 15, wherein the pharmaceutically-acceptable salt of the biodegradable polyamine comprises a polysaccharide, a protein, a peptide, a recombinant protein, a synthetic polyamine, a protamine, a branched polyamine, or an amine-modified natural polymer.

17. The composition of claim 16, wherein the pharmaceutically-acceptable salt of the biodegradable polyamine comprises gelatin modified with an alkyldiamino compound.

18. The composition of claim 9, wherein the polycationic salt comprises a pharmaceutically-acceptable salt of a protamine.

19. The composition of claim 9, wherein the polycationic salt is a pharmaceutically-acceptable salt of salmine or clupein.

20. The composition of claim 9, wherein the polycationic salt is a pharmaceutically-acceptable salt of natural polymer or a synthetic polymer containing two or more guanidinyl sidechains.

21. The composition of claim 9, wherein the polycationic salt comprises a pharmaceutically-acceptable salt of a polyacrylate comprising two or more pendant guanidinyl groups.

22. The composition of claim 9, wherein the polycationic salt comprises a pharmaceutically-acceptable salt of a homopolymer comprising pendant guanidinyl groups.

23. The composition of claim 9, wherein the polycationic salt comprises a pharmaceutically-acceptable salt of a copolymer comprising two or more pendant guanidinyl groups.

24. The composition of claim 9, wherein the polycationic salt comprises a pharmaceutically-acceptable salt of a synthetic polyguanidinyl copolymer comprising an acrylate, methacrylate, acrylamide, or methacrylamide backbone and two or more guanidinyl groups pendant to the backbone.

25. The composition of claim 9, wherein the polycationic salt comprises a pharmaceutically-acceptable salt of a synthetic polyguanidinyl copolymer comprising the polymerization product between a monomer selected from the group consisting of an acrylate, a methacrylate, an acrylamide, a methacrylamide, or any combination thereof and a pharmaceutically-acceptable salt of compound of formula I 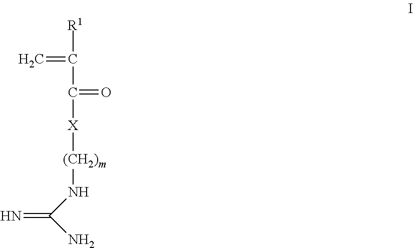
wherein R¹ is hydrogen or an alkyl group, X is oxygen or NR⁵, where R⁵ is hydrogen or an alkyl group, and m is from 1 to 10.

26. The composition of claim 25, wherein the polycationic salt comprises a copolymerization product between the compound of formula I and an acrylate, a methacrylate, an acrylamide, or a methacrylamide,

27. The composition of claim 25, wherein the polycationic salt comprises a copolymerization product between the compound of formula I and methacrylamide, N-(2-hydroxypropyl)methacrylamide (HPMA), N-[3-(N′-dicarboxymethyl)aminopropyl]methacrylamide (DAMA), N-(3-aminopropyl)methacrylamide, N-(1,3-dihydroxypropan-2-yl) methacrylamide, N-isopropylmethacrylamide, N-hydroxyethylacrylamide (HEMA), or any combination thereof.

28. The composition of claim 25, wherein R¹ is methyl, X is NH, m is 3.

29. The composition of claim 25, wherein the mole ratio of the guanidinyl monomer of formula Ito the comonomer is from 1:20 to 20:1.

30. The composition of claim 25, wherein the polyguanidinyl copolymer has an average molar mass from 1 kDa to 1,000 kDa.

31. The composition of claim 1, wherein the polyanionic polyelectrolyte is derived by dissolving a polyanionic salt in water.

32. The composition of claim 31, wherein the polyanionic salt comprises a pharmaceutically-acceptable salt of a synthetic polymer or a naturally-occurring polymer.

33. The composition of claim 31, wherein the polyanionic salt comprises two or more carboxylate, sulfate, sulfonate, borate, boronate, phosphonate, or phosphate groups.

34. The composition of claim 31, wherein the polyanionic salt comprises a pharmaceutically-acceptable salt of a glycosaminoglycan or an acidic protein.

35. The composition of claim 34, wherein the glycosaminoglycan comprises chondroitin sulfate, heparin, heparin sulfate, dermatan sulfate, keratin sulfate, or hyaluronic acid.

36. The composition of claim 31, wherein the polyanionic salt comprises a pharmaceutically-acceptable salt of a protein having a net negative charge at a pH of 6 or greater.

37. The composition of claim 31, wherein the polyanionic salt comprises a pharmaceutically-acceptable salt of a polymer comprising anionic groups pendant to the backbone of the polymer, incorporated in the backbone of the polymer backbone, or a combination thereof.

38. The composition of claim 31, wherein the polyanionic salt comprises a pharmaceutically-acceptable salt of a homopolymer or copolymer comprising two or more anionic groups.

39. The composition of claim 31, wherein the polyanionic salt is a copolymer comprising two or more fragments having the formula XI 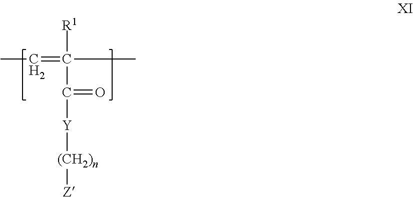
wherein R⁴ is hydrogen or an alkyl group;
n is from 1 to 10;
Y is oxygen, sulfur, or NR³⁰, wherein R³⁰ is hydrogen, an alkyl group, or an aryl group;
Z′ is a pharmaceutically-acceptable salt of an anionic group.

40. The composition of claim 39, wherein Z′ is carboxylate, sulfate, sulfonate, borate, boronate, a substituted or unsubstituted phosphate or phosphonate.

41. The composition of claim 40, wherein n is 2.

42. The composition of claim 31, wherein the polyanionic salt comprises a polyphosphate.

43. The composition of claim 42, wherein the polyphosphate comprises a natural polymer or a synthetic polymer.

44. The composition of claim 42, wherein the polyphosphate comprises polyphosphoserine.

45. The composition of claim 42, wherein the polyphosphate comprises a polyacrylate comprising two or more pendant phosphate groups.

46. The composition of claim 42, wherein the polyphosphate is the copolymerization product between a phosphate acrylate and/or phosphate methacrylate with one or more additional polymerizable monomers.

47. The composition of claim 31, wherein the polyanionic salt has from 10 to 1,000 phosphate groups.

48. The composition of claim 31, wherein the polyanionic salt comprises a pharmaceutically-acceptable salt of an inorganic polyphosphate, an organic polyphosphate, or a phosphorylated sugar.

49. The composition of claim 48, wherein the polyanionic salt comprises a pharmaceutically-acceptable salt of inositol hexaphosphate.

50. The composition of claim 48, wherein the polyanionic salt comprises a hexametaphosphate salt.

51. The composition of claim 48, wherein the polyanionic salt comprises sodium hexametaphosphate.

52. The composition of claim 31, wherein the polyanionic salt comprises a pharmaceutically-acceptable salt of cyclic inorganic polyphosphate, a linear inorganic polyphosphate, or a combination thereof.

53. The composition of claim 31, wherein the polyanionic salt comprises a pharmaceutically-acceptable salt of a polyacrylate comprising two or more pendant phosphate groups.

54. The composition of claim 31, wherein the polyanionic salt comprises a pharmaceutically-acceptable salt of the copolymerization product between a phosphate or phosphonate acrylate or phosphate or phosphonate methacrylate with one or more additional polymerizable monomers.

55. The composition of claim 1, wherein the composition further comprises a reinforcing component, wherein the reinforcing component comprises natural or synthetic fibers, water-insoluble filler particles, a nanoparticle, or a microparticle.

56. The composition of claim 55, wherein the reinforcing component comprises natural or synthetic fibers, water-insoluble filler particles, a nanoparticle, or a microparticle.

57. The composition of claim 1, wherein the composition further comprises one or more bioactive agents, wherein the bioactive agent comprises an antibiotic, a pain reliever, an immune modulator, a growth factor, an enzyme inhibitor, a hormone, a messenger molecule, a cell signaling molecule, a receptor agonist, an oncolytic virus, a chemotherapy agent, a receptor antagonist, a nucleic acid, a chemically-modified nucleic acid, or any combination thereof.

58. The composition of claim 1, wherein the composition has a viscosity of from 10 cp to 20,000 cp.

59. The composition of claim 1, wherein the total positive/negative charge ratio of the polycationic polyelectrolytes to the polyanionic polyelectrolytes is from 4 to 0.25 and the ion concentration in the composition is from 0.5 M to 2.0 M.

60. The composition of claim 1, wherein the concentration of the polycationic polyelectrolytes and the polyanionic polyelectrolytes is sufficient to yield a charge ratio of polycationic polyelectrolytes to polyanionic polyelectrolytes from 0.5:1 to 2:1.

61. The composition of claim 1, wherein the composition has a pH of 6 to 9.

62. An injectable composition produced by the method comprising mixing at least one polycationic salt, at least one polyanionic salt, and a transient contrast agent in water, wherein the polycationic salt dissociates into polycationic polyelectrolytes and anionic counterions, and the polyanionic salt dissociates into polyanionic polyelectrolytes and cationic counterions, wherein the composition has an ion concentration that is (i) sufficient to prevent association of the polycationic polyelectrolytes and the polyanionic polyelectrolytes in water and (ii) greater than the concentration of ions in a subject, whereupon introduction of the composition into the subject a solid is produced in situ, and the transient contrast agent diffuses out of the solid.

63. A method for producing a solid in a subject in situ comprising introducing into the subject the composition in any one of claims 1-62, wherein upon introduction of the composition into the subject the composition is converted to a solid in situ.

64. A method for producing a bioactive eluting depot in the subject comprising injecting into the subject the composition in any one of claims 1-62.

65. A method for reducing or inhibiting blood flow in a blood vessel of a subject comprising introducing into the vessel the composition in any one of claims 1-62, whereupon introduction of the composition into the vessel the composition is converted to a solid in situ within the vessel.

66. The method of claim 65, wherein the method reduces or inhibits blood flow to a tumor, an aneurysm, a varicose vein, a vascular malformation, or a bleeding wound.

67. The method of claim 65, wherein the method reinforces the inner wall of a blood vessel in the subject.

68. A kit comprising
(a) a composition comprising a mixture of at least one polycationic salt and at least one polyanionic salt,
(b) a transient contrast agent, and
(c) instructions for making the injectable composition in any one of claims 1-62,
wherein the polycationic salt dissociates into polycationic polyelectrolytes and anionic counterions, and the polyanionic salt dissociates into polyanionic polyelectrolytes and cationic counterions, wherein the composition has an ion concentration that is (i) sufficient to prevent association of the polycationic polyelectrolytes and the polyanionic polyelectrolytes in water and (ii) greater than the concentration of ions in a subject, whereupon introduction of the composition into the subject a solid is produced in situ, and the transient contrast agent diffuses out of the solid.

69. The kit of claim 68, wherein the composition comprising the mixture of the at least one polycationic salt and the at least one polyanionic salt is a dry powder.

70. The kit of claim 68, wherein the composition comprising the mixture of the at least one polycationic salt and the at least one polyanionic salt further comprises water.

71. The kit of claim 68, wherein the contrast agent is present in water.

72. A kit comprising
(a) at least one polycationic salt,
(b) at least one polyanionic salt,
(c) a transient contrast agent, and
(d) instructions for making the injectable composition in any one of claims 1-62.
wherein the polycationic salt dissociates into polycationic polyelectrolytes and anionic counterions, and the polyanionic salt dissociates into polyanionic polyelectrolytes and cationic counterions, wherein the composition has an ion concentration that is (i) sufficient to prevent association of the polycationic polyelectrolytes and the polyanionic polyelectrolytes in water and (ii) greater than the concentration of ions in a subject, whereupon introduction of the composition into the subject a solid is produced in situ, and the transient contrast agent diffuses out of the solid.
